(19) United States
(12) Patent Application Publication
(10) Pub. No.: US 2012/0149791 A1
(43) Pub. Date: Jun. 14, 2012

(54) OLIGOMERIC PRODUCTS OF POLYETHYLENE TEREPHTHALATE (PET) AND METHODS OF MAKING AND USING SAME

(76) Inventors: Kristopher M. Felice, Wolverine Lake, MI (US); George F. Willard, Ravenswood, WV (US); Eric W. Uffman, Maryland Heights, MO (US); Albert C. Lee, Creve Coeur, MO (US)

(21) Appl. No.: 13/024,281

(22) Filed: Feb. 9, 2011

Related U.S. Application Data
(60) Provisional application No. 61/337,568, filed on Feb. 9, 2010.
(60) Provisional application No. 61/404,621, filed on Oct. 6, 2010.

(51) Int. Cl. C08J 11/24; C07C 69/82; C07C 31/20; C08G 63/91; C09D 167/02

(52) U.S. Cl. 521/48.5; 525/437; 524/605; 568/852; 560/85

(57) ABSTRACT

The presently disclosed and claimed inventive concept(s) relates generally to the method of producing a resin replacement compound, the resin replacement compound itself, and the use of the resin replacement compound. More particularly, but not to be construed as limiting, the presently disclosed and claimed inventive concept(s) relate to the recovery of an oligomeric form of polyethylene terephthalic acid from waste polyethylene terephthalate. In one particular aspect, the presently disclosed and claimed inventive concept(s) relate to the recovery of an oligomeric form of polyethylene terephthalic acid from waste products, such as beverage containers, made from polyethylene terephthalate. In an additional aspect, the presently disclosed and claimed inventive concept(s) relate to the use of a recovered oligomeric form of polyethylene terephthalic acid in a toner composition.

### CROSS REFERENCE TO RELATED APPLICATIONS

The present application claims the benefit under 35 U.S.C. 119(e) of U.S. Provisional Application Ser. No. 61/337,568, filed Feb. 9, 2010, the entirety of which is hereby expressly incorporated herein by reference. The present application also claims the benefit under 35 U.S.C. 119(e) of U.S. Provisional Application Ser. No. 61/404,621, filed Oct. 6, 2010, the entirety of which is hereby expressly incorporated herein by reference.

### BACKGROUND OF THE INVENTION

1. Field of the Disclosed and Claimed Inventive Concepts

The presently disclosed and claimed inventive concept(s) relates generally to the method of producing a resin replacement compound, the resin replacement compound itself, and the use of the resin replacement compound. More particularly, but not to be construed as limiting, the presently disclosed and claimed inventive concept(s) relate to the recovery of an oligomeric form of polyethylene terephthalic acid from waste polyethylene terephthalate. In one particular aspect, the presently disclosed and claimed inventive concept(s) relate to the recovery of an oligomeric form of polyethylene terephthalic acid from waste products, such as beverage containers, made from polyethylene terephthalate. In an additional aspect, the presently disclosed and claimed inventive concept(s) relate to the use of a recovered oligomeric form of polyethylene terephthalic acid in a toner composition.

2. Background

Most polyester resins used in commercial applications are formed from raw materials which are rising in price and have relatively large markets. Accordingly, recovery of these raw materials from scrap, waste and used products is an important economical consideration as well as an ecological consideration. One widely used polyester is polyethylene terephthalate (hereinafter “PET”) made from terephthalic acid and ethylene glycol. Additionally, a Bisphenol A polyester resin could be used in a manner similar to PET.

Over the past 20 years, there has been an increased push throughout the world to increase recycling of polyester resins. Plastic bottles commonly used for drinks and carbonated beverages, for example, are made from polyethylene terephthalate and represent a large potential source of recoverable polyesters: either as bulk refined PET or the terephthalic acid and ethylene glycol monomers that constitute PET. It is estimated that from 375 to 500 million pounds of polyethylene terephthalate were used for beverage bottles in 1980, for example. More recently, more than 2.4 billion pounds of plastic bottles were recycled in 2008. Although the amount of plastic bottles recycled in the U.S. has grown every year since 1990, the actual recycling rate remains steady at around 27 percent. Recent legislation in several states requiring a deposit refundable upon return of all empty beverage containers has established an ongoing procedure for collecting and separating polyethylene terephthalate containers which must be recycled or otherwise disposed of in an economical manner. Additionally, many municipalities have implemented voluntary or mandatory recycling programs in conjunction with trash pickup and disposal.

PET beverage containers cannot be reused since the elevated temperatures required for sterilization deforms the container. PET containers can, however, be ground into small pieces for use as a filler material or remelted for formation of different articles. Such recycled material may be referred to interchangeably herein as “recycled PET”, “scrap PET”, “waste PET”, and/or “rPET”. The polyethylene terephthalate recovered by such processes contains impurities, such as pigment, paper, other undesirable polymers and metal from caps. Consequently, applications for polyethylene terephthalate reclamation by mechanical means are limited to non-food uses and low purity molded products.

In the past, several different techniques have been proposed for recovering pure or isolated terephthalic acid and ethylene glycol monomers from waste polyethylene terephthalate. One known technique involves, for example, the depolymerization of polyethylene terephthalate by saponification. Saponification is the hydrolysis of an ester under basic conditions to form an alcohol and the salt of a carboxylic acid (carboxylates).

In one known approach for saponification, polyethylene terephthalate is reacted with an aliphatic alcohol and a dialkyl terephthalate is recovered. This approach is exemplified in U.S. Pat. Nos. 3,321,510, 3,403,115 and 3,501,420, all of which are hereby incorporated by reference in their entirety. In a second known approach, polyethylene terephthalate is reacted with an aqueous solution of an alkali metal hydroxide or carbonate (usually sodium hydroxide) at an elevated temperature to yield a water soluble salt of terephthalic acid and ethylene glycol. The reaction product is acidified to liberate terephthalic acid which is water insoluble and the terephthalic acid precipitate is separated by filtration or the like. This approach is exemplified by U.S. Pat. Nos. 3,377,519, 3,801,273 and 3,956,088, all of which are hereby incorporated by reference in their entirety. U.S. Pat. No. 3,544,622, the entire contents of which is hereby incorporated by reference in its entirety, similarly discloses a variation to previously known approaches wherein the reaction is carried out under conditions to produce a water insoluble salt of terephthalic acid which is separated, washed and then acidified to produce terephthalic acid. Additional patents have also been issued on various improvements to these processes, such as U.S. Pat. Nos. 5,414,107, 5,223,544, 5,328,982, 5,045,122, 5,710,315, 5,532,404, 6,649,792, 6,723,873, 6,255,547, 6,580,005, 6,075,163, 7,173,150, 6,770,680, 7,098,299, and 7,338,981, the entire contents of each of which are hereby incorporated by reference in their entirety.

Empty beverage containers obtained from consumers may have aluminum caps lined with polyvinyl chloride or the like, wrap around polypropylene coated paper labels bonded to the surface with a polyvinyl acetate adhesive, residual sugars and, in some cases, polyethylene base caps for strengthening purposes. Without costly controls, reaction conditions in the saponification processes disclosed in the above-noted patents tend to cause some dissolution of these extraneous materials which then become impurities in the recovered terephthalic acid and require costly purification. Therefore, various approaches have been considered for removing these materials from the containers prior to grinding or separating them from the polyethylene terephthalate after grinding. Such separation procedures represent a significant increase in the overall cost of recovery as well as an energy inefficient means of recycling the waste PET. Thus, while such saponification methodologies for the recycling of PET into its monomer constituents are generally considered to be successful, it is an expensive and economically inefficient way in which to obtain such monomers for producing new PET polymers for use.

Polyester resins are unsaturated resins formed by the reaction of dibasic organic acids and polyhydric alcohols. Among their many uses are sheet moulding compound, bulk moulding compound and the toner of laser printers. Unsaturated polyesters are condensation polymers formed by the reaction of polyols (also known as polyhydric alcohols, i.e., organic compounds having multiple alcohol or hydroxy functional bonds). Typical polyols used in making unsaturated polyesters include glycols and ethylene acids such as phthalic acid and maleic acid. Unsaturated polyesters differ from saturated polyesters in that acids or glycols having double bond unsaturation are included in the polymer which also lowers the viscosity of the resin to a useable extent. Typically, polyester resins are thermosetting, i.e., the plastic softens when initially heated, but sets permanently rigid once it has cooled or has been chemically cured.

As mentioned, polyester resins are used in the production of toner for laser printers. In general, and as used herein, the term “toner” refers to a powder used in laser printers and photocopiers to form the printed text and images on the paper. In its early form, toner was simply carbon powder. In order to improve the quality and other characteristics of the toner output, carbon powder (such as carbon black) was melt-mixed with one or more polymers, including polyester resin. Typically, toner particles are melted by the heat of the fuser within the laser printer and are bound to the surface of the paper.

As is now known, commercial laser printing utilizing toner particles is performed by a variety of methods. All methods require, however, that the toner particles be charged, deposited on a desired area of the paper, and conveyed to/through a heat source that causes the toner particles to melt and fuse uniformly to the paper.

In order to have uniform particle charging and thereby attain acceptable print quality, each particle must be uniform. From a raw material standpoint, this means that the material chosen for use must be uniform. In addition, the toner must be properly mixed to ensure the contents of the toner are uniformly distributed as well. Charge control agents, for example, magnetite, are added to the toner before mixing/extruding in order to increase the particles' ability to uniformly distribute charge. In one methodology, for example, the raw materials may be extruded using a twin screw extruder in order to mix the raw materials properly and ensure uniform mixing and distribution of the particles for use in a toner.

The specific polymer used in toner varies by manufacturer but can be a styrene acrylate copolymer, a polyester resin, a styrene butadiene copolymer, other specialty polymers, and combinations thereof. Toner formulations vary from manufacturer to manufacturer and even from machine to machine. Typically formulation, granule size and melting point have the highest variation among manufacturers.

Originally, the particle size of toner averaged 14-16 micrometres or greater. In order to improve image resolution, particle size was reduced, eventually reaching about 8-10 μm for 600 dots per inch resolution. Further reductions in particle size producing further improvements in resolution are being developed through the application of new technologies such as Emulsion-Aggregation. Toner manufacturers maintain a quality control standard for particle size distribution in order to produce a powder suitable for use in their printers.

Toner has traditionally been made by compounding the ingredients and creating a slab which was broken or pelletized, then turned into a fine powder with a controlled particle size range by air jet milling. This process results in toner granules with varying sizes and aspherical shapes. In order to get a finer print, some companies are using chemical processes to grow toner particles from molecular reagents. This results in more uniform size and shapes of toner particles. The smaller, uniform shapes permit more accurate color reproduction and more efficient toner use.

While the basic chemical structure and nature of toner has been widely studied and known for some time, the ability to use recycled polyester resins within toner has eluded researchers and commercial entities. To date, there is no commercially available toner comprising recycled resins of any appreciable amount.

### SUMMARY OF THE INVENTION

The presently claimed and disclosed inventive concept(s) provide for a simple and efficient process for recovering oligomeric raw materials from polyester waste products in economical yields and high purity form for use as resin replacements. In one embodiment, the process is a saponification process. In a second embodiment, the process is a glycolysis process for recovering polyethylene terephthalate oligomers in economical yields from used polyethylene terephthalate beverage containers. The high purity terephthalic acid oligomers can thereafter be used as resin replacements or resin extenders within existing systems or products requiring the use of resins and, more particularly, polyester resins. An example of the use of such polyethylene terephthalate oligomers as a polyester resin replacement/extender would be toner for use in laser printers.

### BRIEF DESCRIPTION OF THE DRAWING

FIG. 1 is a modulated differential scanning calorimetry (“mDSC”) analysis of the white precipitate from the first reaction. The derivative of the heat capacitance (J/(g° C.°)) indicated the presence of a melt at 120.95° C., indicating the presence of a small amount of oligomer.

FIG. 2 is liquid chromatography-mass spectrometry analysis of fully digested rPET.

FIG. 3 is a 19× magnification of a portion of laser printed text using (a) commercially available toner; (b) toner produced according to Formula 1; and (c) toner produced according to Formula 10.

FIG. 4 details liquid chromatography-mass spectrometry analysis of digested rPET having a MW distribution of 277-656 daltons.

FIG. 5 is a Fourier Transform Infrared Spectroscopy (FTIR)/Hyperion Reflectance analysis of the white precipitate from the first reaction.

FIG. 6 is an mDSC analysis of the white precipitate from the second reaction. The derivative of the non-reversible capacitance shows copious amounts of reactivity.

FIG. 7 is a Fourier Transform Infrared Spectroscopy (FTIR)/Hyperion Reflectance library match of FIG. 5. The top spectrum is of the white precipitate shown in FIG. 5, the matching bottom spectrum is Amoco TA-12, Terephthalic Acid.

FIG. 8 is an mDSC analysis of the white precipitate from the second reaction. The linear derivative of the non-reversible heat capacitance and reversible heat flow indicates that the white precipitate has reacted completely.

FIG. 9 shows the GPC data that illustrates that the rPET has been digested from a MW of 41,253 to a MW of 338-369 daltons.

FIG. 10 is a graphical representation of the effect on reaction time by the addition of water.

FIG. 11 is a graphical representation of the effect on reaction time by varying the amount of catalyst (i.e., zinc acetate) used in the reaction.

FIG. 12 is a graphical representation of the effect on reaction time versus the amount of catalyst.

FIG. 13 is a graphical representation of the effect of predigested rPET (Sample Ref. No. 188-17) on the digestion of green rPET.

FIG. 13A is a graphical representation of the effect of predigested rPET on the digestion of green rPET.

FIG. 14 is a graphical representation of the effect of predigested rPET (Sample Ref. No. 733-12, MW=1004) in a rPET digestion reaction.

FIG. 15 is a graphical representation of the effect of predigested rPET (Sample Ref. No. 188-17, MW=3381) in a rPET digestion reaction.

FIG. 16 is a graphical representation of the effect of predigested rPET on a clear rPET digestion.

FIG. 17 is a graphical representation of ¹H NMR spectra of an isolated white solid obtained in accordance with the present disclosure.

FIG. 18 is a graphical representation of ¹H NMR spectra over a 8.0-8.2 ppm range of an isolated white solid obtained in accordance with the present disclosure.

FIG. 19 is a graphical representation of ¹H NMR spectra over a 4.2-5.1 ppm range of an isolated white solid obtained in accordance with the present disclosure.

FIG. 20 is a graphical representation of ¹H NMR spectra over a 3.2-3.8 ppm range of an isolated white solid obtained in accordance with the present disclosure.

FIG. 21 is a graphical representation of ¹³C NMR spectra of an isolated white solid obtained in accordance with the present disclosure.

FIG. 22 is a graphical representation of ¹³C NMR spectra over a 128-160 ppm range of an isolated white solid obtained in accordance with the present disclosure.

FIG. 23 is a graphical representation of ¹³C NMR spectra over a 58-68 ppm range of an isolated white solid obtained in accordance with the present disclosure.

FIG. 24 is a graphical representation of ¹H NMR spectrum of an isolated white solid plus 2 drops of ethylene glycol obtained in accordance with the present disclosure.

FIG. 25 is a graphical representation of ¹H NMR spectrum of an isolated white solid plus 2 drops of ethylene glycol obtained in accordance with the present disclosure.

FIG. 26 is a graphical representation of ¹H NMR spectrum of an isolated white solid plus 2 drops of ethylene glycol obtained in accordance with the present disclosure.

FIG. 27 is a graphical representation of ¹H NMR spectrum of ethylene glycol in DMSO-d6.

FIG. 28 is a graphical representation of ¹H NMR spectrum of ethylene glycol in DMSO-d6.

FIG. 29 is a graphical representation of ¹H NMR spectrum of DMSO-d6 Blank.

FIG. 30 is a graphical representation of ¹H NMR spectrum of DMSO-d6 Blank.

FIG. 31 is a graphical representation of ¹H NMR spectrum over a 0-12 ppm range of DMSO-d6 Blank.

FIG. 32 is a graphical representation of ¹H NMR spectra over a 6-12 ppm range of a green semi-solid isolated from green toluene filtrate in accordance with the present disclosure.

FIG. 33 is a graphical representation of ¹H NMR spectra over a 6.8-8.8 ppm range of a green semi-solid isolated from green toluene filtrate in accordance with the present disclosure.

FIG. 34 is a graphical representation of ¹H NMR spectra over a 4.1-5.4 ppm range of a green semi-solid isolated from green toluene filtrate in accordance with the present disclosure.

FIG. 35 is a graphical representation of ¹H NMR spectra over a 2.8-4.0 ppm range of a green semi-solid isolated from green toluene filtrate in accordance with the present disclosure.

FIG. 36 is graphical representation of ¹H NMR spectra over a 1.8-2.7 ppm range of a green semi-solid isolated from green toluene filtrate in accordance with the present disclosure.

FIG. 37 is a graphical representation of ¹H NMR spectra over a 1.25-1.95 ppm range of a green semi-solid isolated from green toluene filtrate in accordance with the present disclosure.

FIG. 38 is a graphical representation of ¹H NMR spectra over a 0-1 ppm range of a green semi-solid isolated from green toluene filtrate in accordance with the present disclosure.

FIG. 39 is a graphical representation of ¹H NMR spectra over a 0-107 ppm range of a green semi-solid isolated from green toluene filtrate in accordance with the present disclosure.

FIG. 40 is a graphical representation of ¹³C NMR spectra over a 0-220 ppm range of a green semi-solid isolated from green toluene filtrate in accordance with the present disclosure.

FIG. 41 is a graphical representation of ¹³C NMR spectra over a 140-220 ppm range of a green semi-solid isolated from green toluene filtrate in accordance with the present disclosure.

FIG. 42 is a graphical representation of ¹³C NMR spectra over a 80-140 ppm range of a green semi-solid isolated from green toluene filtrate in accordance with the present disclosure.

FIG. 43 is a graphical representation of ¹³C NMR spectra over a 112-140 ppm range of a green semi-solid isolated from green toluene filtrate in accordance with the present disclosure.

FIG. 44 is a graphical representation of ¹³C NMR spectra over a 10 -75 ppm range of a green semi-solid isolated from green toluene filtrate in accordance with the present disclosure.

FIG. 45 is a graphical representation of ¹³C NMR spectra over a 56-75 ppm range of a green semi-solid isolated from green toluene filtrate in accordance with the present disclosure.

FIG. 46 is a graphical representation of ¹³C NMR spectra over a 13-34 ppm range of a green semi-solid isolated from green toluene filtrate in accordance with the present disclosure.

FIG. 47 is a graphical representation of ¹³C NMR spectra over a 28-30 ppm range of a green semi-solid isolated from green toluene filtrate in accordance with the present disclosure.

FIG. 48 is a pictorial representation of a printed sheet using a toner prepared in accordance with the present disclosure.

### DESCRIPTION OF THE PREFERRED EMBODIMENTS

Before explaining at least one embodiment of the presently disclosed and claimed inventive concept(s) in detail, it is to be understood that the presently disclosed and claimed inventive concept(s) is not limited in its application to the details of construction, experiments, exemplary data, and/or the arrangement of the components set forth in the following description or illustrated in the drawings. The presently disclosed and claimed inventive concept(s) is capable of other embodiments or of being practiced or carried out in various ways. Also, it is to be understood that the phraseology and terminology employed herein is for purpose of description and should not be regarded as limiting.

Broadly, the process of the presently claimed and disclosed inventive concept(s) includes the step of reacting polyethylene terephthalate scrap and/or waste with ethylene glycol containing a catalyst at an elevated temperature and at atmospheric pressure for a sufficient time to decrease the molecular weight of the PET scrap to an oligomeric state. In one embodiment, the catalyst is a zinc acetate catalyst capable of decreasing the amount of activation energy for depolymerization of polyethylene terephthalate. In an additional step, precipitated PET oligomer is recovered from the reaction mixture and dried. The dried PET oligomer can thereafter be used as a resin replacement or resin extender, e.g., as a replacement for the polyester resin commonly found in toner used in laser printers.

While the process of the presently disclosed and claimed inventive concept(s) can be used to treat a wide variety of polyethylene terephthalate scrap or waste, it is particularly adaptable for processing used polyethylene terephthalate beverage containers. As used hereinafter, the term “scrap” means scrap, waste and/or used products of polyethylene terephthalate. The term “scrap” also includes within its definition varying sizes and shapes of waste and/or used products of polyethylene terephthalate. Scrap PET, as used herein, may include whole products made of PET (e.g., a beverage bottle) or further processed products made of PET. In one embodiment, the further processing includes the chipping or shredding of PET products in order to produce a scrap PET material suitable for use in the disclosed methodologies. Alternatively, the further processing may include nitrogen jet milling of the PET products in order to produce a scrap PET material having an average size of about 10 microns. One of ordinary skill in the art would appreciate that the further processing step may include a multitude of processing steps including, but not limited to, pin milling, jet milling, media milling, rolling and crushing, all of which would be understood to fall within the broad disclosure presented herein.

In one embodiment, further processing of the PET raw material is accomplished via milling. For example, recycled bulk PET (rPET) having a particle size in the range of 100-200 microns was obtained from Clean Tech Incorporated (Dundee, Mich.). This bulk recycled PET is formed from PET plastic bottles that are sorted by color, ground, washed and repelletized under vacuum conditions to restore molecular weight. The recycled bulk PET was in the form of grayish pellets. Further processing for this embodiment entailed liquid nitrogen jet milling of the recycled bulk PET pellets according to the conditions outlined in Table 1 and performed by LiquaJet/The Jet Pulverizer Co. (Moorestown, N.J.). The processing steps performed by LiquaJet are proprietary methods kept as a trade secret by the company. Generally, the material was milled with liquid nitrogen in order to obtain a product having a desired state. The results of particle size shown in Table 1 were determined on a Wet Horiba Ri:1.5750 (HORIBA Ltd., Austin, Tex.).

TABLE 1

| | Avg. Particle | |
|---|---|---|
| | Size of rPET | |
| Test Condition | in microns. | Grit |
| 1A | 64.4 | NO |
| 1B | 59.1 | NO |
| 2A | 33.1 | NO |
| 2B | 83.0 | NO |
| 3A (2A 2X) | 27.4 | NO |
| 4A | 51.6 | NO |
| 4B | 51.6 | YES |
| 5A | 108.5 | YES |
| 5B | 153.0 | YES |

After a second milling using the liquid nitrogen jet milling process, the rPET material was found to have an average size of 27.4 microns. Further processing (i.e., additional liquid jet milling steps) would achieve a specification of a rPET material having an average size of less than about 33 microns and, more particularly, from about 7 to about 10 microns. Although such small sizes of rPET can be obtained, it was found that the process(es) according to the presently disclosed and claimed inventive concept(s) do not require such a small starting size of the rPET. Rather, it was found that rPET having a size of from about 25 microns to about 100 microns can be used and, more particularly, rPET scrap having a size of from about 50 microns to about 100 microns. Such sizes should not be considered as limiting, however, as the presently disclosed and claimed inventive concept(s) have been found to be suitably applied to rPET scrap having a size equal to or greater than 200 microns.

Chemical recycling of a plastic is by definition the recycling of a chemical by partially altering its chemical structure through a chemical reaction into a viable material. PET is produced by the reaction of ethylene glycol and terephthalic acid. The reaction between the acid and the glycol results in an ester linkage and water. Water is removed from the reaction kettle to continue polymerization thereby increasing the average molecular weight of the PET.

The depolymerization of the rPET into a reactive, lower melting point (mp) material was accomplished according to novel methodologies of the presently disclosed and claimed inventive concept(s). The molecular weight of the polymer is reduced until a molecular weight of 280-680 is achieved, for example. At this molecular weight the PET has the physical qualities of a lower melting point (mp) and increased reactivity useful for some applications.

An initial reaction scheme for depolymerization of the rPET into a reactive lower melting point material according to the presently disclosed and claimed inventive concept(s) utilized the reactant weights given in Table 2.

TABLE 2

| Reactants | Amount | |
|---|---|---|
| Deionized Water | 300 | ml |
| rPET (TABLE A-1A) | 48.4393 | g |
| Anhydrous Sodium Acetate | 3.0263 | g |
| 50% by Weight Sodium Hydroxide | 30 | ml |

The reactants were all weighed and added to the reaction kettle. The kettle was set up with a stir bar and a condenser with cold water running through it. A heating mantle was used to heat the mixture as it was stirring. The temperature of the reaction kettle was maintained at a constant temperature of from about 150-175° C. The reaction was allowed to proceed for 6 hours at which time the rPET had completely dissolved. The reaction solution was then cooled to a uniform highly basic solution. A small sample of the cooled mixture was placed into a beaker and concentrated hydrochloric acid was added until a white precipitate formed.

More particularly, the procedure for the reaction of Table 2 was as follows:
1. 250 ml of water was added to the reaction kettle.
2. 48.4393 g of rPET (Table 1-Sample 1A) was added to the reaction kettle.
3. 3.0262 g of anhydrous sodium acetate catalyst was added to the reaction kettle.
4. 30 ml of 50% by weight sodium hydroxide was added.
5. The reaction was heated to a temperature of 150-175° C.
6. Water (350 mL) was added in two increments.
7. When the rPET was completely dissolved (after approximately 6 hours), the reaction kettle was allowed to cool to room temperature.
8. An aliquot of the solution was removed from the kettle and acidified with concentrated hydrochloric acid which afforded a white precipitate.

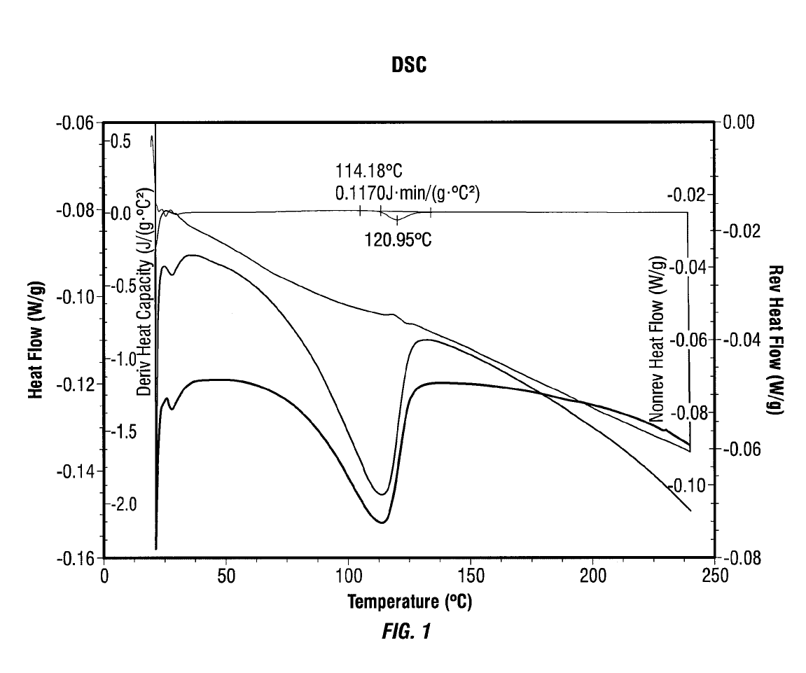

The white precipitate was dried and characterization by differential scanning calorimetry (DSC), FTIR, and LC-MS (Liquid Chromatography with Mass Spectrometric detection) revealed that the resultant products were primarily the monomers: ethylene glycol and terephthalic acid. Modulated DSC (mDSC) was used to determine the material's melting point. A small melt occurring at 109° C. indicated, moreover, that a minor amount of bis(2-hydroxyethyl)terephthalate (i.e., a glycol-terephthalic acid-glycol trimer) residually remained in the white precipitate (FIG. 1, mDSC heat flow). This melting point of 109° C. is consistent with a commercially available bis(2-hydroxyethyl) terephthalate material supplied by Sigma Aldrich Co. (CAS# 959-26-2). The reaction scheme outlined above and in Table 2 proved that an atmospheric pressure based system could be used to depolymerize rPET into oligomers of rPET, i.e., a resin replacement or extender composition (indicated by the 109° C. melt on the mDSC). LC-MS analysis indicated, however, that the reaction according to Table I had proceeded to near completion. The chemical structure and molecular weight analysis indicated that the majority of the white precipitate was actually terephthalic acid or monomer, not the rPET oligomer(s) desired. As such, the reaction conditions were modified in order to obtain greater quantities of oligomeric rPET having varying molecular weights.

The depolymerization process according to Table 2 is believed to proceed via basic hydrolysis of the ester linkages in the rPET in basic conditions using a sodium acetate catalyst. In order to synthesize a precipitated product that could be used, for example, in a toner composition, the reactants of Table 2 were manipulated. The manipulation of the reactants was undertaken to change the ratio of terephthalic acid to ethylene glycol in the final product. The average molecular weight (MW) of the rPET used for these reactions was ˜41,000 daltons which is greater than the range required for a resin extender. For a toner product, for example, but not by way of limitation, the average molecular weight of the polyester resin found in common formulations is from about 800 daltons to about 100,000 daltons or more. As such, the MW of the rPET oligomers according to at least one embodiment of the presently disclosed and claimed inventive concept(s) should be in the range of from about 280 to about 41,000 daltons and, more particularly, in the range of from about 280 to about 680 daltons.

In order to obtain a low molecular weight oligomeric digested rPET species, a reaction was designed such that every additional mole of ethylene glycol (EG) added would be capable of reducing the molecular weight of the polymer through transesterification. Exemplary reactions were carried out in which 5 equivalents of ethylene glycol was added for every mole of terephthalic acid. These exemplary reactions are summarized in Table 3 and were carried out in accordance with the methods given for the initial reaction set forth in Table 2.

TABLE 3

| | Reaction 2 | Reaction 3 |
|---|---|---|
| Ethylene glycol (g) | 620 | 620 |
| PET (FW = 192 g/mol) (g) | 384 | 384 |
| Moles PET | 2 | 2 |
| ZnOAc (g) | 10 | 20 |
| Moles EG Added | 10 | 10 |

The exemplary reactions given in Table 3 were considered to be complete once the pellets of rPET were completely dissolved and the reaction reached a homogeneous, liquid phase. In each case, this required approximately 6 hours. The precipitates formed from Reaction #2 and Reaction #3 were added directly to reagent grade ethyl alcohol (Ethyl Alcohol CAS 64-17-5 90%, Methyl Alcohol CAS 67-56-1 5%,Isopropyl Alcohol CAS 67-63-0 5%) and made into a slurry. The slurries were then dried in an oven at 32° C. for 24 hrs. These resultant white products were then characterized analytically.

In each reaction, the recovered and dried precipitates comprised oligomeric units of rPET, i.e., the recovered and dried precipitates were primarily composed of incompletely digested oligomers of rPET. The mDSC analysis of the precipitates samples, according to the exemplary reactions of Table II (FIG. 1), showed that the material had reacted completely.

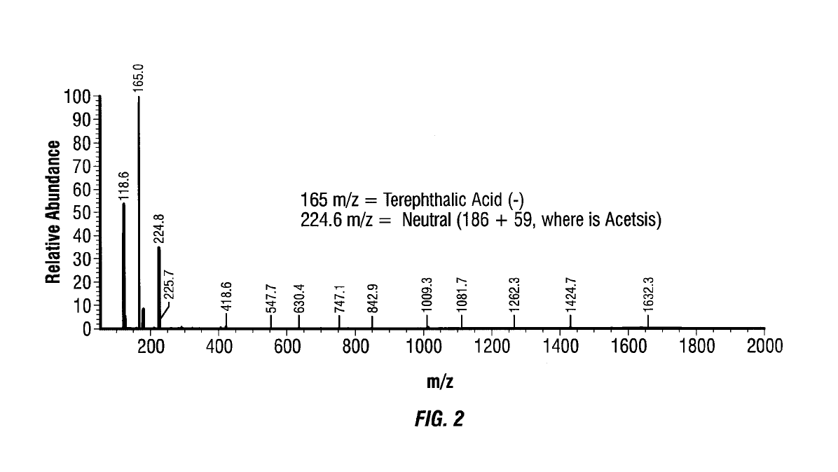

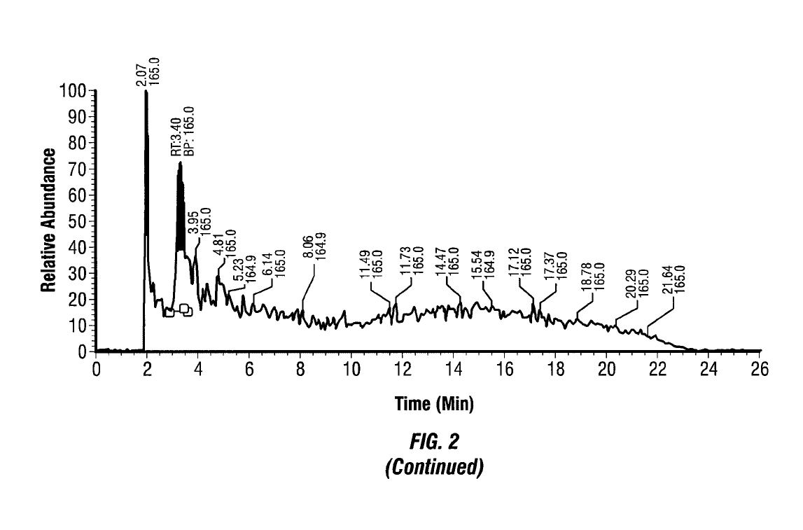

GPC characterization of the molecular weight distribution was also performed. The white product collected from these reactions was analyzed by LC/MS and Gel Permeation Chromatography to determine the molecular weight distribution of the product mixture. LC/MS analysis of the product (FIG. 2) shows three main peaks in the chromatogram. The first broad peak with a maximum at 9.81. minutes gives a mass to charge ratio consistent with the ionized forms of the trimer species. The second peak having an elution time of 21.54 minutes has a mass to charge ratio of 508. The third and smaller peak eluting at 27.24 minutes has a mass to charge ratio of 656. Although there are other peaks that have much lower UV absorbances, these are the three major, non-solvent peaks presented in the chromatograms. This analysis was repeated on three separate preparations of the material with little to no variability of the results and demonstrates that the molecular weight distribution of the material produced by these reactions is in the range of 277-656 daltons.

As is common for GPC analysis of PET, the samples were analyzed in comparison to polystyrene MW calibration standards. Duplicate preparations of the digested rPET were analyzed. The results are listed in Table 4:

TABLE 4

| | | | | | Poly- |
|---|---|---|---|---|---|
| | Mn | MW | Mz | Mp | dispersity |
| Undigested Scrap PET | 11,802 | 41,253 | 72,290 | 42,204 | 3.50 |
| Digested PET | 282 | 338 | 417 | 300 | 1.20 |
| Duplicate preparation | 304 | 369 | 461 | 334 | 1.21 |

As shown in Table 4, the molecular weight of the rPET has been reduced from an MW of 41,253 to an MW of 338-369. The very low polydispersity values of 1.20 and 1.21 indicate that the material is highly uniform in its molecular weight distribution. Further, the high level of agreement in the two preparations of material indicates that the procedure is repeatable and consistent. The GPC results are also in strong agreement with the LC/MS data that shows the largest peak for oligomers in the range of 277 daltons and smaller contributions for those in the 508 and 656 ranges.

Additional experiments were conducted to determine additional boundaries of the experimental design and the differentiation/production of rPET oligomers having differing molecular weights, the use of different sources and types of rPET as well as reaction kinetics and parameters. The variables studied were: (1) lowering the ratio of ethylene glycol to rPET, (2) the addition of water to the reaction, (3) differing amounts of catalyst used, (4) the use of additives, (5) the reaction time, (6) the reaction temperature, and (7) the use of mixed digestions (i.e., green and clear rPET).

While the foregoing has been described in conjunction with rPET (i.e., recycled polyethylene terephthalate), it is contemplated and has been experimentally determined (as shown hereafter) that virgin PET (vPET—i.e., polyethylene terephthalate that has not previously been molded into a product, a previously molded PET product that has not been commercially used, a previously molded PET product that has been used to hold a product or act as packaging but has not been put into commercial streams of commerce, combinations of the above, etc.) can be used in the described methods. As such, the term rPET should be understood as encompassing polyethylene terephthalate material having a recycled content of from 0% to 100% and still be within the scope of the described and claimed invention(s) herein.

Digestion of Virgin PET (Experimental Designator: 749-114): A 1 L, 4-neck flask was equipped with a mechanical stirrer, thermocouple, condenser and stopper. Neopentyl glycol (129.3 g, Aldrich 538256 lot# 07304DHV) was added to the flask and melted. All of the solids dissolved when the flask was at 95° C. (internal temperature). Zinc acetate dihydrate (3.85 g, Alfa Aesar 11559, lot # C11W013) was added in one portion. The temperature was increased to 135° C. and virgin PET (i.e., vPET—240 g, Poly Sciences 04301 lot #46418) was added in portions over a 15 min period. The temperature was raised to 200° C. and held for 4.5 h. The pellets dissolved to give a slightly hazy solution—i.e., dPET obtained from a reaction of vPET. The resulting dPET from vPET was observed to have a hydroxyl number of 354 (over an average of three determinations) which corresponds to 6.31 mmol/g, while the viscosity was measured to be 1416 centipoise (cP) at 80° C. GPC data indicated that the resulting dPET from vPET had an average MW of 1237 and the resulting chromatograph was similar to dPET from a rPET source that was digested in a similar manner. Overall, the data for virgin digested material was consistent with material prepared from recycled PET using the same stoichiometry.

### Scaling of rPET Digestion Reactions

In order to examine the ability of the rPET digestion to be scaled to commercial production scale, a series of experiments were conducted to approximate such commercial conditions.

Experimental Designator 188-73: A 22 L 4-neck flask was fitted with a Teflon stir blade connected to a high-torque overhead stirrer, thermocouple and condenser. Neopentyl glycol (3651 g, 35 mol Aldrich 538256-3KG Lot 10134519) was added to the flask and melted at 145 deg. Zinc acetate dihydrate (109 g, 0.5 mol Alfa Aesar 11559 Lot A13U005) was added in portions over 2 min. Recycled PET (6743 g, 35 mol on the basis of the monomer, green pellets) were added in portions over 1 h 40 min as the set point of the temperature controller was increased to 200° C. after the final addition of rPET. The temperature was held at 200° C. until all of the pellets dissolved (approximately 4.7 h). After all of the pellets of rPET had dissolved, the solution was allowed to cool and the resulting product was packaged at approximately 72° C. Approximately 10.5 kg of digested PET product was produced.

Experimental Designator 749-74: A 22 L 4-neck flask was fitted with a Teflon stir blade connected to a high-torque overhead stirrer, thermocouple, stopper and condenser. Neopentyl glycol (3651 g, 35 mol Aldrich 538256-3 KG Lot 07304DHV) was added to the flask and melted at 155° C. Zinc acetate dihydrate (109 g, 0.5 mol Alfa Aesar 11559 Lot A13U005) was added in portions over 2 min. Recycled PET (6744 g, 35 mol on the basis of the monomer, green pellets) were added in portions over 1 h 15 min. The set point of the temperature controller was increased incrementally to 200° C. The temperature was held at 200° C. until all of the pellets dissolved (approximately 3.75 h). After all of the pellets of rPET had dissolved, the solution was allowed to cool and packaged at approximately 80° C. Approximately 10.5 kg of digested PET product was produced and GPC indicated an average MW of 1389. As used herein, the term “molecular weight” or “MW” in reference to PET is defined as the peak average molecular weight (Mₚ) as determined by Gel Permeation Chromatography (GPC).

Experimental Designator Lymtal PP: A 175 gallon stainless steel reactor, fitted with a condenser, was charged with neopentyl glycol (399.4 lbs) and heated to 260° F. After the alcohol was melted, zinc acetate dihydrate (11.93 lbs) was added. After it dissolved, green recycled PET (737.7 lbs) was added in equal portions over 30 mins. The temperature was increased ° to 378° F. over approximately 4 h. The solids required 8 hours at 350 to 378° F. to completely dissolve. Upon cooling to 176° F., approximately 1124 lbs of digested PET product was produced and GPC indicated an average MW of 1386.

### Effect of Lowering the Ratio of Ethylene Glycol to rPET for Digestion of the rPET

A series of digestion experiments were performed to study the effect of reducing the relative amount of ethylene glycol used in the rPET digestions. Reactions were set up in 1 L round bottom flasks with a mechanical stirrer, thermocouple, temperature controller, heating mantle and condenser. The flasks were charged with rPET obtained from Clean Tech of Dundee, Mich., ethylene glycol (Aldrich “Reagent Plus” grade, the CoA indicated >99% by GC, no mention of water content in either the CoA or the Product Specification Sheet) and zinc acetate dihydrate. The reaction was performed with both clear and green rPET. The results of these experiments are summarized in Table 5.

TABLE 5

| | Molar ratio | | | rxn | |
|---|---|---|---|---|---|
| Ref. | ethylene glycol | rPET | Zn(OAc)2 | time | Notes |
| 185-2 | 2.79 | 1.00 | 0.014 | 1 h | Clear PET, MP = 733 Pl = 1.111 |
| 188-7-1 | 2.12 | 1.00 | 0.014 | 30 min | Clear PET, MP = 715 Pl = 1.131 |
| 188-1-1 | 2.14 | 1.00 | 0.014 | 1 h | Clear PET, MP = 742 Pl = 1.129 |
| 188-5-1 | 2.01 | 1.00 | 0.014 | 35 min | Clear PET, MP = 752 Pl = 1.132 |
| 188-10 | 1.73 | 1.00 | 0.014 | 1 h | Clear PET, MP = 731 Pl = 1.147 |
| 188-12 | 1.73 | 1.00 | 0.014 | 5 h | Clear PET |
| 188-9 | 1.06 | 1.00 | 0.014 | 13 h | Clear PET |
| 188-17 | 2.79 | 1.00 | 0.014 | 57 min | Green PET: MW 715, 1091, 1430 |
| 188-18-1 | 1.50 | 1.00 | 0.014 | 51 min | Green PET: MW 684, 1048, 1336 |
| 188-18-2 | 1.00 | 1.00 | 0.014 | 24 min | Green PET: MW 701, 1082, 1386 |

The digestion experiments with clear rPET showed a general trend of longer reaction times when decreasing amounts of ethylene glycol was used. The digestion experiments with green rPET showed a general trend of a faster reaction rate with decreasing amounts of ethylene glycol.

GPC analyses were performed on the green rPET digestions. The data showed that the molecular weight distributions were consistent, regardless of whether or not clear or green rPET was used. It was also observed that the differences in the amount of ethylene glycol used in this series of reactions appeared to have a minimal impact on MW distribution. The differences in the physical characteristics of the product were more pronounced, i.e., reducing the amount of ethylene glycol produced a less waxy solid digested rPET, for example.

Further efforts towards lowering the amount of ethylene glycol required to digest the rPET were also performed. For example, the use of additives to facilitate mixing was studied. Reactions were performed with 155 g clear rPET, 30 mL ethylene glycol and either (a) 30 g of predigested rPET material from sample Ref. No. 188-5, or (b) 30 g of bis(2-hydroxyethyl)terephthalate (BHTA) as additives. Zinc acetate was added to two of the three reactions. The results are summarized in Table 6.

TABLE 6

| | molar ratio | | | | |
|---|---|---|---|---|---|
| | ethylene | | | | rxn |
| Ref. | glycol | rPET | Zn(OAc)₂ | other | time |
| 188-13 | 0.67 | 1.00 | 0.014 | 16% BHTA | 30 min |
| 188-11 | 0.67 | 1.00 | 0.014 | 16% 188-5 | 12 min |
| 188-14 | 0.67 | 1.00 | 0 | 16% 188-5 | 33 min |

Reactions were also performed with green rPET. In sample Ref. No. 188-19, the reaction pot was charged with 310 g green rPET, 60 mL ethylene glycol, 2.0 g zinc acetate dehydrate and 30 g of predigested rPET product from sample Ref. No. 188-17. The reaction time was 30 min as measured from the time the pot was >170° C. until the solids were dissolved. The results are summarized in Table 7.

TABLE 7

| | molar ratio | | | | |
|---|---|---|---|---|---|
| | ethylene | | | | rxn |
| Ref. | glycol | rPET | Zn(OAc)₂ | Other | time |
| 188-19 | 0.67 | 1.00 | 0.006 | 9.7% 188-17 | 30 min |
| 188-20 | 0.67 | 1.00 | 0.014 | 9.7% BHTA | 4.5 h |

Sample Ref. No. 188-20 was similar to sample Ref. No. 188-19 except that 30 g of bis(2-hydroxyethyl)terephthalate was the additive instead of predigested rPET of Sample Ref. No. 188-17. The time for the reaction to reach completion was for Sample Ref. No. 188-20 longer at 4.5 h. It is believed that the bis(2-hydroxyethyl)terephthalate chelated the Zn⁺² ions thereby increasing the time of the reaction.

These digestion experiments demonstrate that the amount of ethylene glycol used to digest the rPET material can be demonstrably lowered.

### Effect of Adding Water to rPET Digestion

The effect of adding water to the digestion of rPET was studied by running a set of reactions in 1 L round bottom flasks set up as described above for the digestion of rPET with lower amounts of ethylene glycol. The flasks were charged with rPET (obtained from Clean Tech), ethylene glycol (Aldrich “reagent plus” grade, the CoA indicated >99% by GC, no mention of water content in either the CoA or the Product Specification Sheet) and zinc acetate dihydrate. Water was added in the following amounts: 0 mL, 0.75 mL, 1.25 mL and 2.50 mL. Quantities and molar ratios of the constituents are shown in Table 8 and the results of the experiments are summarized in Table 9.

TABLE 8

| | MW | quantity, g | mol | Eq |
|---|---|---|---|---|
| Ethylene glycol | 62.07 | 192.0 | 3.09 | 1.93 |
| PET (ester), MW = monomer | 193 | 310.0 | 1.61 | 1.00 |
| zinc acetate | 219.51 | 5.00 | 0.023 | 0.014 |

TABLE 9

| Ref. | % H₂O wrt Zn(OAc)₂ | Reaction time (min) |
|---|---|---|
| 188-1-1 | 0% | 60 |
| 188-1-3 | 15% | 75 |
| 188-1-2 | 25% | 120 |
| 185-3 | 50% | 180 |

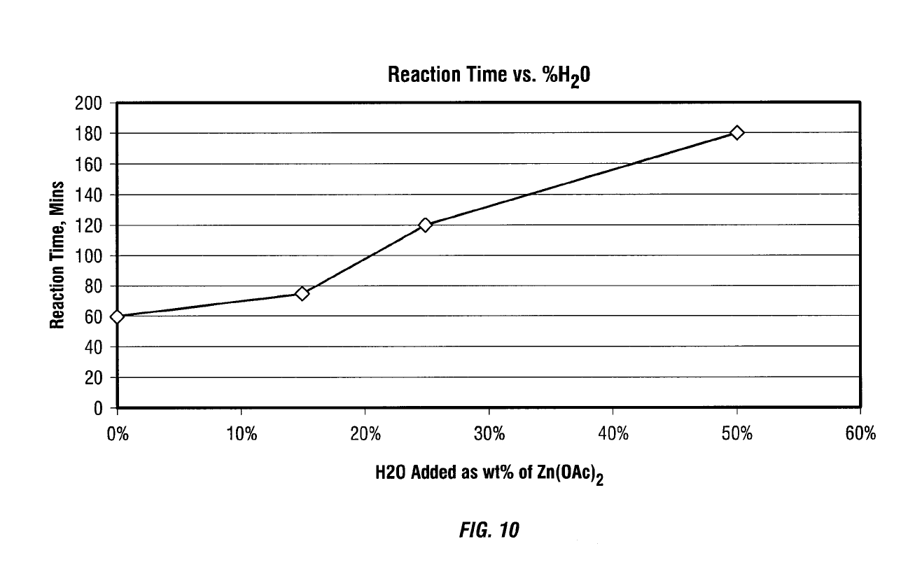

The mixtures were stirred and heated with a set point of 200° C. on the controllers and full power going to the heating mantles. Reaction times were determined from the time when the pot reached a hold temperature of ca. 180-200° C. until a solution formed (solids fully dissolved). The reaction times are shown in Table 9. The data showed that the addition of water inhibited the progress of the reaction and that the greater the amount of water added, the longer the reaction time. FIG. 10 shows the reaction time for digesting rPET as a function of increased percentage water in the reaction mixture.

### Effect of Different Amounts of Zinc Acetate Catalyst on the Digestion of rPET

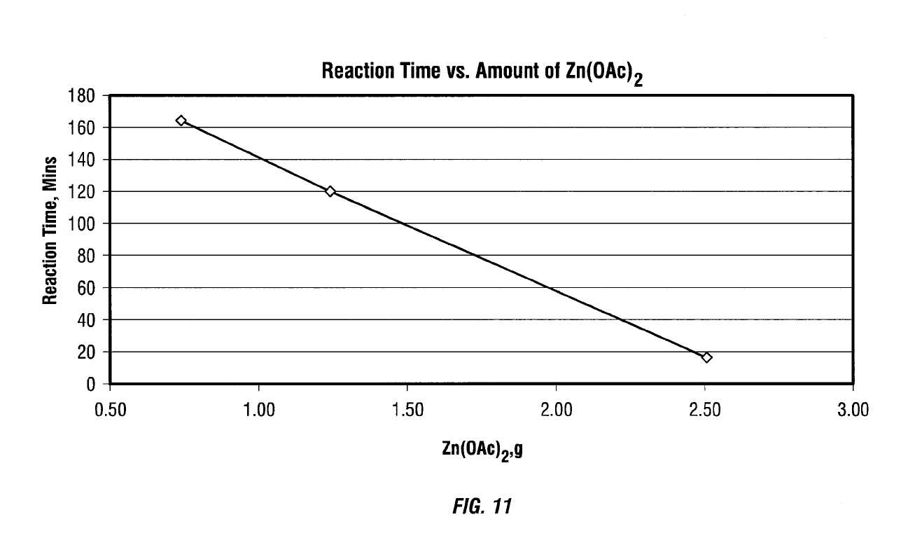

A series of reactions were performed with varying amounts of catalyst to determine the time necessary for reaction completion. The experiments were set up in 1 L flasks as described above with respect to the experiments determining the amount of ethylene glycol required, provided: 310 g clear rPET and 180 mL ethylene glycol. The flasks were heated to 180-195° C. with full power setting on the temperature controller. Zinc acetate (Zn(OAc)₂) was prepared as a mixture in ethylene glycol. The quantities were 2.5 g, 1.25 g and 0.75 g in 10 mL ethylene glycol and the mixtures were sonicated for 15-20 min to aid in dissolving the solids. The 2.5 g Zn(OAc)₂ preparation had some insoluble material present. The catalysts were added to the hot reaction mixtures as a bolus. Reaction times for completion, based on consumption of PET, are summarized in Table 10. FIG. 11 shows the reaction time for digesting rPET as a function of the increased amount of catalyst (Zn(OAc)₂) in the reaction mixture.

TABLE 10

| Ref. | Zn(OAc)₂, g | time, min |
|---|---|---|
| 188-3-1 | 2.50 | 16 |
| 188-3-2 | 1.25 | 120 |
| 188-3-3 | 0.75 | 165 |

As can be appreciated from FIG. 11, a linear relationship exists between the amount of catalyst (i.e., zinc acetate) used in the reaction and the reaction time. The reaction rate appeared to be unusually fast when 2.50 g Zn(OAc)₂ was added, i.e., it was shorter than the time observed when twice the amount of catalyst was used in previous experiments. Factors that may have influenced this are the differences between temperature controllers, characteristics of the mantle or predissolving the catalyst in ethylene glycol and adding it to a hot mixture of rPET and ethylene glycol.

In order to determine if predissolving zinc acetate had any effect, the reactions described above were repeated with:
1. 5.0 g zinc acetate slurried in 10 mL ethylene glycol;
2. 1.88 g zinc acetate dissolved in 10 mL ethylene glycol; and
3. 5.0 g zinc acetate added in neat.

The reaction mixtures were heated to 180° C. and the catalyst was added. The results are summarized in Table 11.

TABLE 11

| Ref. | Zn(OAc)₂ | Reaction time, min |
|---|---|---|
| 188-5-1 | 5.00 g in 10 mL ethylene glycol | 38 |
| 188-5-2 | 1.88 g in 10 mL ethylene glycol | 130 |
| 188-5-3 | 5.00 g added neat | 65 |

The reaction time with zinc acetate slurried in ethylene glycol was almost twice as fast as what was observed when the catalyst was used neat. The reaction time for the experiment using 5.0 g of zinc acetate was still longer than that observed when 2.5 g zinc acetate was used. This can probably be attributed to differences in equipment and/or atmospheric conditions, for example. As one skilled in the art will appreciate, the reaction time decreases as the amount of catalyst increases.

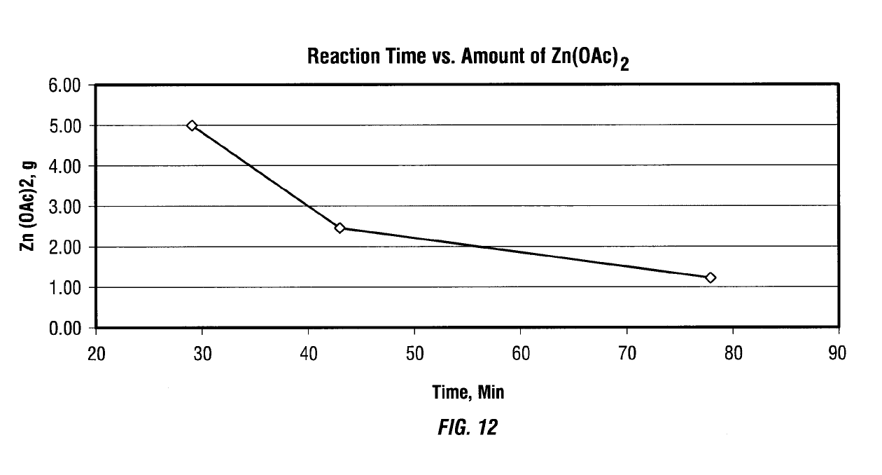

In an effort to ensure uniformity in the temperature of the pot at the start of the reaction (i.e., the addition of zinc acetate), the series was repeated by refluxing mixtures of rPET and ethylene glycol for approximately 1 h before adding the catalyst. The quantities used and the reaction times are given in Table 12. FIG. 12 graphically displays the results of reaction time versus the amount of Zn(OAc)₂.

TABLE 12

| | | ethylene | | Reaction |
|---|---|---|---|---|
| Ref. | rPET | glycol | Zn(OAc)₂ | time, min |
| 188-7-1 | 310 g | 190 mL | 5.00 g | 29 |
| 188-7-2 | 310 g | 190 mL | 2.50 g | 43 |
| 188-7-3 | 310 g | 190 mL | 1.25 g | 78 |

In summary, it was found that decreasing the amount of catalyst resulted in longer reaction times.

### Use of Additives: Predigested PET for Digestion of rPET

rPET pellets were digested with rPET that had been previously digested in earlier experiments, i.e., the previously digested portions of rPET were investigated for use as a “digesting agent” for the reactions. The “digesting” functionality appears to lower the amount of ethylene glycol used to digest the rPET by serving as the “glycolysis” agent, thereby omitting or decreasing the amount and/or complete use of free ethylene glycol.

In order to determine the feasibility of such a digesting agent, green colored rPET was treated with an amount of the previously digested rPET material produced in sample Ref. No. 188-17. Two reactions were run with the green rPET pellets and previously digested rPET. A third reaction was run with the addition of zinc acetate to the mixture. The results of these experiments are summarized in Table 13.

TABLE 13

| | | Pre- | | rxn | | | |
|---|---|---|---|---|---|---|---|
| | | digested | | temp, | | | |
| Ref. | rPET | PET | Zn(OAc)₂ | ° C. | rxn time | Mp | PI |
| 188-22 | 310 g | 50 g | 0 | 185 | 24 min | 3293 | 1.474 |
| 188-23 | 310 g | 50 g | 0 | 160 | 6 h | 3381 | 1.521 |
| 188-24 | 310 g | 50 g | 5.0 g | 165 | 70 min | 3743 | 1.631 |

Sample Ref. No. 188-22 was initially difficult to stir but when predigested rPET melted, mixing became easier. The reaction mixture was heated to 185° C. and went to completion in 24 min (based on rPET dissolving). Sample Ref. No. 188-23 was similar to sample Ref. No. 188-22 but run at 160-165° C. for 6 h. Mixing this reaction was difficult as a crust formed on top of the reaction mixture before all of the pellets dissolved. Therefore, it was required to be periodically broken up with a spatula. Sample Ref. No. 188-24 was similar to Sample Ref. No. 188-23 with zinc acetate added. The reaction was heated for 70 min at the end of which the liquid portion was hazy but pellets were not evident. GPC analysis of the reactions showed that the resulting digested rPET material had a molecular weight range of 3293 to 3743. Polydispersity values for the resulting material ranged from 1.474 to 1.631.

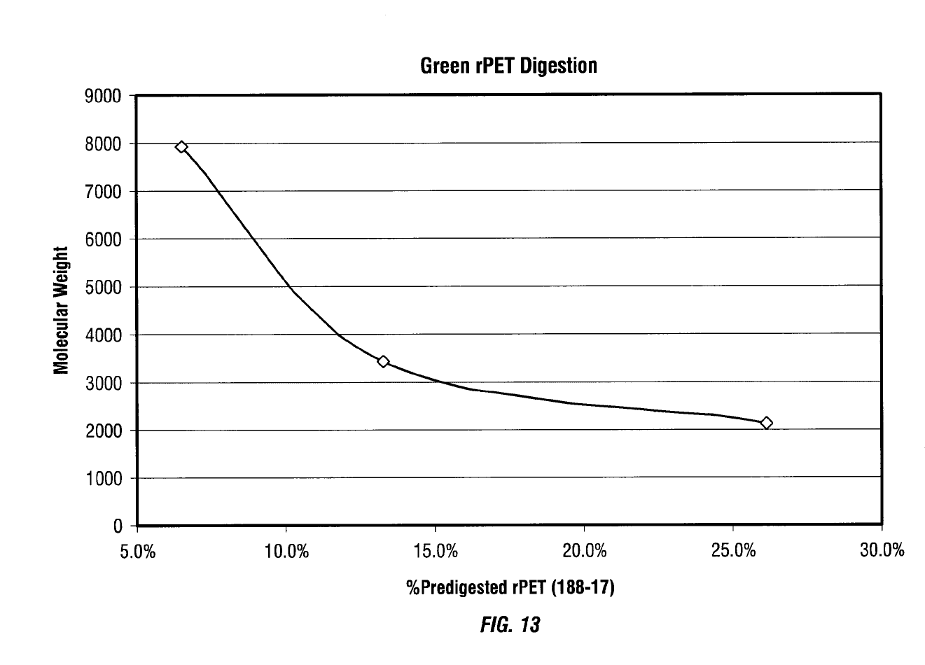

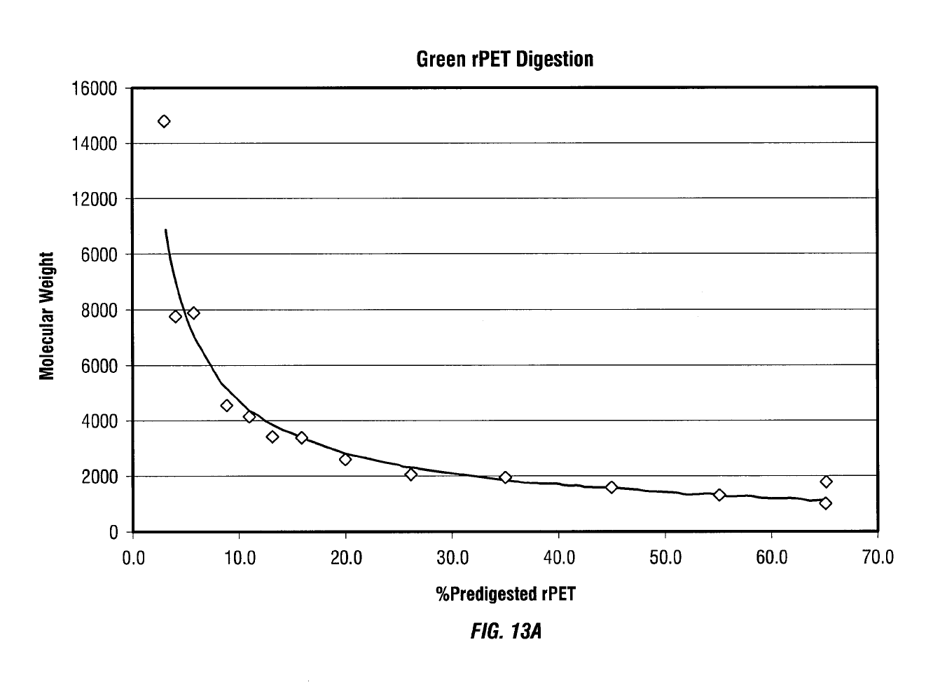

In order to produce digested rPET with different molecular weight ranges, green rPET was heated with varying amounts of previously digested rPET from Sample Ref. No. 188-17 until a solution formed. The quantities used and GPC data are shown in Table 14. The relationship between the percentage of predigested rPET used (i.e., the “predigesting agent” rPET) and the molecular weight produced in the reaction showed a correlation of lower molecular weight digested rPET products being produced with increasing amounts of predigested rPET material being used as shown in FIG. 13.

TABLE 14

| | | % | | |
|---|---|---|---|---|
| | | predigested | | |
| Ref. | Description | PET | Mp | PI |
| 188-26 | green, used predigested 188-17 | 26.1% | 2139 | 1.393 |
| 188-27 | green, used predigested 188-17 | 13.2% | 3425 | 1.519 |
| 188-28 | green, used predigested 188-17 | 6.5% | 7921 | 1.770 |

In order to further explore the range of different molecular weight products that can be produced according to the above-discussed methodology, digestions utilizing 3.3% to 65% predigested rPET were performed. Three different lots of predigested rPET were used in these experiments (all three were produced by heating a mixture of 310 g rPET with 250 mL ethylene glycol and 5.0 g zinc acetate). The experiments using varying amounts of predigested PET and GPC results are shown in Table 15 and in FIG. 13A. As shown, digested rPET having molecular weight ranges of 1058 to 14777 were produced.

TABLE 15

| | | % | | |
|---|---|---|---|---|
| | | predigested | | |
| Ref. | Description | PET | Mp | PI |
| 732-2 | green, used predigested 188-17 | 3.3 | 14777 | 1.908 |
| 733-1 | green, used predigested 188-17 | 4.3 | 7723 | 1.757 |
| 188-28 | green, used predigested 188-17 | 6.5 | 7921 | 1.770 |
| 733-4 | green, used predigested 188-17 | 9.0 | 4575 | 1.657 |
| 733-5 | green, used predigested 188-17 | 11.0 | 4151 | 1.575 |
| 188-27 | green, used predigested 188-17 | 13.2 | 3452 | 1.519 |
| 188-23 | green, used predigested 188-17 | 16.1 | 3381 | 1.631 |
| 188-29 | green, used predigested 188-17 | 20.0 | 2584 | 1.442 |
| 188-26 | green, used predigested 188-17 | 26.1 | 2139 | 1.393 |
| 733-7 | green, used predigested 734-1 | 35.0 | 1951 | 1.398 |
| 733-9 | green, used predigested 734-1 | 45.0 | 1627 | 1.345 |
| 733-11 | green, used predigested 733-10 | 55.0 | 1307 | 1.301 |
| 733-2 | green, used predigested 734-1 | 65.0 | 1058 | 1.261 |
| 732-4 | green, used predigested 188-17 | 65.2 | 1772 | 1.335 |

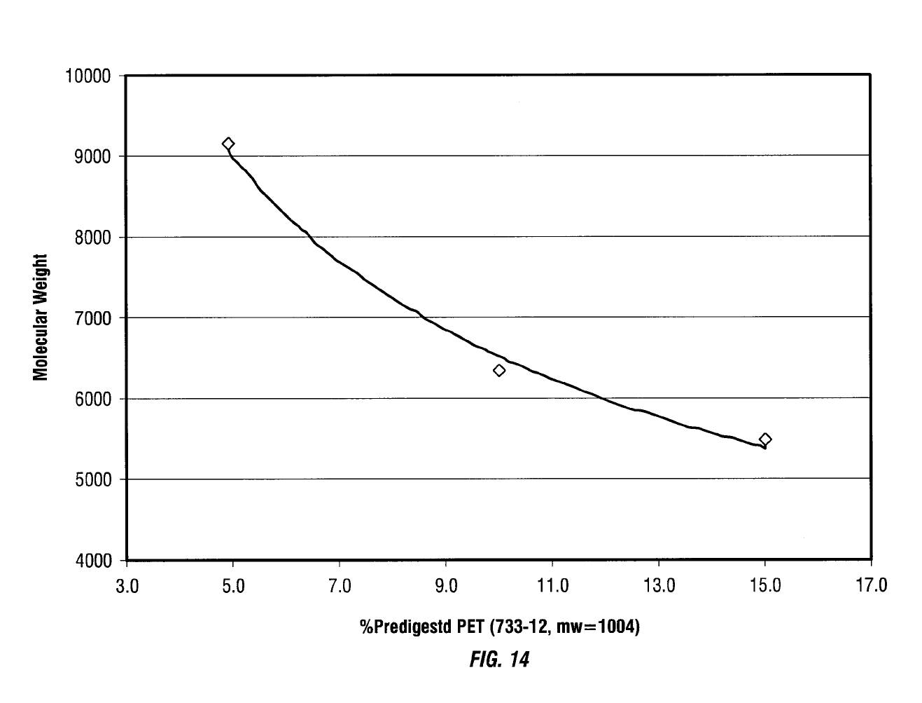

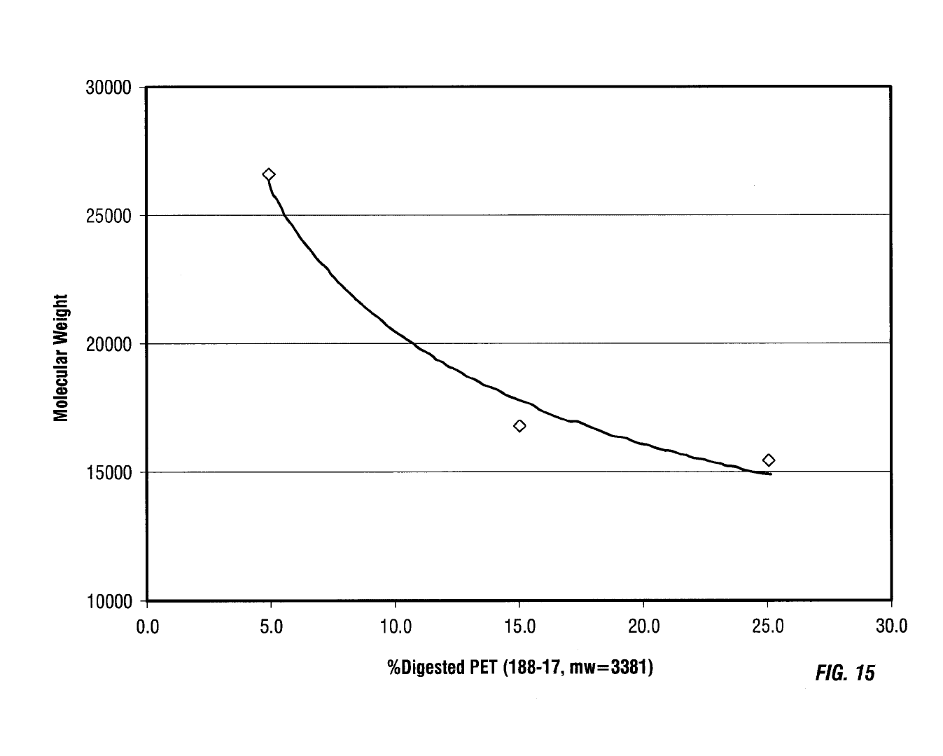

Further experiments were conducted in order to produce digested rPET having a higher molecular weight. In the first set of experiments, green rPET was heated with 5%, 10% and 15% predigested green rPET sample Ref. No. 733-12 (MW 1004). In a second set of experiments, rPET was heated with 5%, 15% and 25% predigested green rPET Sample Ref. No. 188-23 (MW 3381). Both sets of experiments showed that higher molecular weight material was produced when decreasing amounts of predigested rPET material were used, e.g., using 5% predigested rPET material having a MW of 3381. produced a digested rPET material having a MW of 26,535, while 5% predigested rPET having a MW of 1004 produced a digested rPET material having a MW of 9,162. The results of these experiments are summarized in Table 16 and FIGS. 14 and 15.

TABLE 16

| | | % | | |
|---|---|---|---|---|
| | | predigested | | |
| Ref. | Description | PET | Mp | PI |
| 733-15 | green, used predigested 733-12 | 5.0 | 9162 | 1.856 |
| | (MW = 1004) | | | |
| 733-14 | green, used predigested 733-12 | 10.0 | 6342 | 1.751 |
| 733-13 | green, used predigested 733-12 | 15.0 | 5492 | 1.679 |
| 732-12 | green, used predigested 188-23 | 5.0 | 26535 | 2.261 |
| | (MW = 3381) | | | |
| 732-11 | green, used predigested 188-23 | 15.0 | 16765 | 2.133 |
| 732-10 | green, used predigested 188-23 | 25.0 | 15426 | 2.043 |

Additional experiments were also conducted with clear rPET. Clear rPET was heated with predigested rPET Sample Ref. No. 732-3 (732-3 was produced from 310 g rPET, 250 mL ethylene glycol and 5.0 g zinc acetate dehydrate, heated until a solution formed, GPC analysis of Mp=676, PI=1.103). Table 17 summarizes these results.

TABLE 17

| | | % pre- | | | |
|---|---|---|---|---|---|
| | | digested | Line | | |
| Ref. | Description | PET | Voltage | Mp | PI |
| 732-6 | clear, used predigest 732-3 | 3.3 | 100% | 19271 | 2.148 |
| 732-5 | clear, used predigest 732-3 | 4.3 | 100% | 15176 | 2.080 |
| 732-7 | clear, used predigest 732-3 | 9.0 | 100% | 8717 | 1.809 |
| 733-6 | clear, used predigest 732-3 | 11.0 | 100% | 5348 | 1.646 |
| 732-13 | clear, used predigest 732-3 | 35.0 | 47% | 4600 | 1.639 |
| 732-14 | clear, used predigest 732-3 | 45.0 | 47% | 1598 | 1.329 |
| 732-15 | clear, used predigest 732-3 | 55.0 | 47% | 1297 | 1.306 |

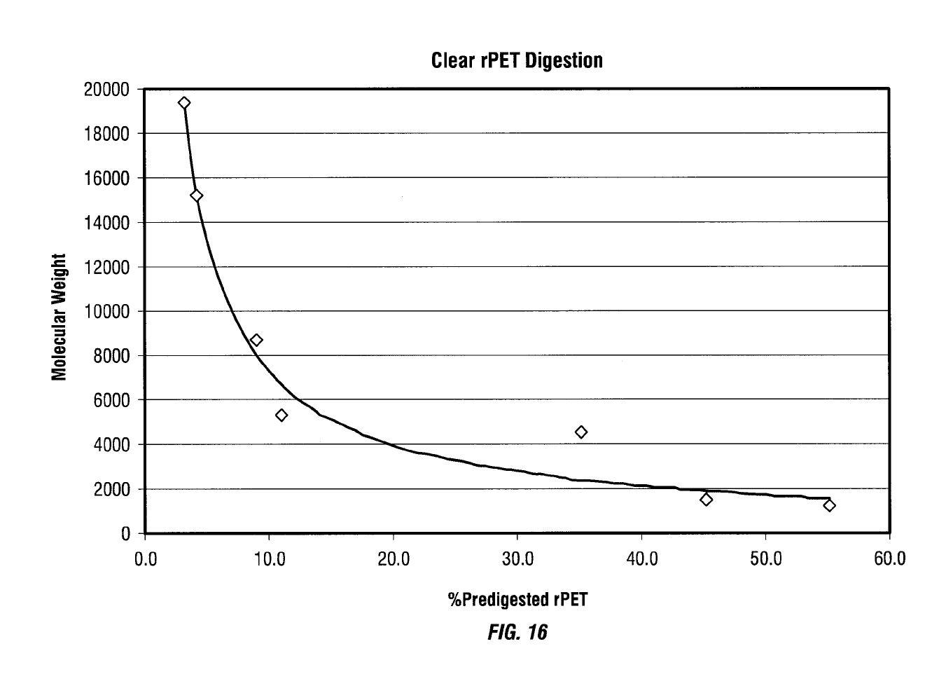

Digested rPET having a molecular weight range of 1,297 to 19,271 was produced and the polydispersity of these materials ranged from 1.306 to 2.148. FIG. 16 is a graphical representation of the percentage predigested rPET used against the molecular weight of the digested rPET produced. The curve depicted in FIG. 16 is similar to what was observed with respect to samples incorporating green rPET.

Samples with Ref. Nos. 732-13, 732-14 and 732-15 were produced by reducing power to the heating source. Instead of providing 100% line voltage to the heating mantle, a variable control power transformer was used to regulate power to 47% of line voltage. (Table 17) Milder heating resulted in higher molecular weight products which is evident in the way that data points fall off the curve at union where the two different heating methods were used. Thus, treating clear rPET with decreasing amounts of predigested rPET material results in higher molecular weight digested rPET material produced.

The use of predigested rPET as a glycolysis agent has, therefore, been proved to be quite effective as a digesting agent and/or as an effective replacement for free ethylene glycol in the reaction mixture. A higher concentration of predigested rPET material gave lower molecular weight digested rPET product.

### In Situ Preparation of 16% “Predigested” rPET

Experiments were performed with both clear and green rPET to determine the effect of an in situ preparation of “predigested” rPET for use as a digesting agent.

For Sample Ref. No. 732-16, 26.1 grams of clear rPET, 0.42 grams of zinc acetate and 23.4 grams of ethylene glycol were heated to reflux, achieving homogeneity at 200° C. 310.0 grams of clear rPET were added to the pre-digested material over 17 minutes, completing the addition at 209° C. Sample Ref. No. 732-16A was taken at an internal thermocouple reading of 262° C. Fifteen minutes elapsed between completing the rPET addition and obtaining homogeneity of the mixture. GPC showed that the resulting material was digested rPET having a MP=3321 and polydispersity of PI=1.510.

For Sample Ref. No. 732-17, 26.1 grams of green rPET, 0.42 grams of zinc acetate and 23.4 grams of ethylene glycol were heated to reflux, achieving homogeneity at 195° C. 310.0 grams of green rPET were added to the pre-digested rPET material over 16 minutes, completing the addition at 199° C. Homogeneity was reached at 0.77 hours after completing the addition of the green rPET. The mixture (Sample Ref. No. 732-17A) reached homogeneity at 214° C. GPC showed that the resulting material was digested rPET having a MP=3614 and polydispersity of PI=1.541.

By way of comparison, Sample Ref. No. 188-22 used 16.1% predigested rPET and produced a digested rPET material having a MP=3293 and polydispersity of PI=1.474. Thus, in situ preparation of “predigested” material gave the same results as using predigested rPET material that was isolated prior to addition to the reaction mixture.

### Effect of Reaction Time and Increased Temperature

Digestion experiments were performed to study the effect of extended reaction times and elevated temperatures of the digested rPET produced. Reactions with extended reaction times were carried out with predigested rPET mixed with rPET pellets as described above but the reaction was held at 190-200° C. for up to 6 h beyond completion. The starting point (t=0) was determined when the rPET pellets went completely into solution. The reaction mixtures were sampled at regular intervals and the pulls were analyzed by GPC.

Tables 18 and 19 summarize the concentrations of predigested rPET which were used and the results of the GPC analysis. When 9% to 65% predigested rPET were used, a long reaction time did not make a difference in the molecular weight of the digested rPET produced (within the limits of the technique). At low concentrations, longer reaction times resulted in lower molecular weight digested rPET products. For instance, the experiment with 3.3% predigested rPET produced a digested rPET product having a MW=19,271 at t=0 and MW=15,407 at t=6 h; the experiment with 4.3% predigested rPET produced a digested rPET product having a MW=15,176 at t=0 and MW=12,854 at t=5.5 h.

TABLE 18

| Ref. | Description | M_{N} | M_{P} | M_{W} | PI |
|---|---|---|---|---|---|
| 733-2A | Green PET digested with 65% | 1162 | 1058 | 1466 | 1.261 |
| | predigested PET (188-17) t = 0 h | | | | |
| 733-2B | t = 1 h | 1162 | 1053 | 1464 | 1.260 |
| 733-2C | t = 2 h | 1160 | 1050 | 1464 | 1.262 |
| 733-2D | t = 3 h | 1170 | 1053 | 1476 | 1.261 |
| 733-2E | t = 4 h | 1146 | 1034 | 1447 | 1.264 |
| 733-2F | t = 5 h | 1137 | 1021 | 1438 | 1.264 |
| 733-2G | t = 6 h | 1133 | 1015 | 1431 | 1.263 |
| 733-4A | Green PET digested with 9% | 3352 | 5185 | 5646 | 1.684 |
| | predigested PET (188-17) t = 5 h | | | | |
| 733-4B | t = 4 h | 3286 | 5106 | 5490 | 1.671 |
| 733-4C | t = 3 h | 3229 | 4917 | 5321 | 1.648 |
| 733-4D | t = 2 h | 3188 | 4780 | 5239 | 1.643 |
| 733-4E | t = 1 h | 3074 | 4612 | 5073 | 1.650 |
| 733-4F | t = 0 h | 3014 | 4575 | 4994 | 1.657 |
| 733-4G | t = 6 h | 3342 | 5287 | 5636 | 1.686 |

TABLE 19

| Ref. | Description | M_{N} | M_{P} | M_{W} | PI |
|---|---|---|---|---|---|
| 732-6A | Clear PET digested with 3.3% | 9513 | 19271 | 20430 | 2.148 |
| | predigested PET (732-3) t = 0 h | | | | |
| 732-6B | t = 1 h | 9580 | 19169 | 21590 | 2.254 |
| 732-6C | t = 2 h | 9248 | 18779 | 21497 | 2.324 |
| 732-6D | t = 3 h | 9357 | 18374 | 21510 | 2.299 |
| 732-6E | t = 4 h | 8813 | 16739 | 21010 | 2.384 |
| 732-6F | t = 5 h | 8506 | 15823 | 20391 | 2.397 |
| 732-6G | t = 6 h | 8254 | 15407 | 20137 | 2.440 |
| 732-5A | Clear PET digested with 4.3% | 7785 | 15176 | 16190 | 2.080 |
| | predigested PET (732-3) t = 0 h | | | | |
| 732-5B | t = 0.5 h | 7839 | 14627 | 15643 | 1.996 |
| 732-5C | t = 1.5 h | 8359 | 16932 | 19093 | 2.284 |
| 732-5D | t = 2 h | 8403 | 16625 | 19605 | 2.333 |
| 732-5E | t = 2.5 h | 8237 | 16561 | 19796 | 2.404 |
| 732-5F | t = 3.75 h | 7669 | 15013 | 19189 | 2.502 |
| 732-5G | t = 4.75 h | 7201 | 14236 | 18899 | 2.624 |
| 732-5H | t = 5.5 h | 6945 | 12854 | 17562 | 2.529 |
| 733-5A | Clear PET digested with 11% | 2848 | 4151 | 4484 | 1.575 |
| | predigested PET (732-3) t = 0 | | | | |
| 733-5B | t = 1 h | 2882 | 4178 | 4527 | 1.570 |
| 733-5C | t = 2 h | 2864 | 4261 | 4568 | 1.595 |
| 733-5D | t = 3 h | 2896 | 4332 | 4620 | 1.595 |
| 733-5E | t = 4 h | 2923 | 4478 | 4736 | 1.620 |
| 733-5F | t = 5 h | 2971 | 4482 | 4821 | 1.623 |
| 733-5G | t = 6 h | 2989 | 4618 | 4915 | 1.644 |

The effect of elevated reaction temperatures was further analyzed by performing a digestion reaction with 100.0 grams of clear rPET with 3.3% predigested clear rPET. Homogeneity was achieved 2.92 hours from the time that heat was applied to the clear rPET. The longer time required to reach homogeneity was a function of the attempt to control the temperature and heating rate. Three samples of the homogeneous mixture were taken and GPC results tabulated:
1. 216° C. internal temperature (Ref. No. 732-18A, time 1235), MP=21171 PI=2.088
2. 249° C. internal temperature (Ref. No. 732-18B, time 1419), MP=19616 PI=2.185
3. 272° C. internal temperature (Ref. No. 732-18C, time 1530), MP=15459 PI=2.231

Thus, reaction time has little, if any, effect on the molecular weight of the product when concentrations of predigested rPET are ≧9%. Low concentrations of predigested rPET, however, indicate that the molecular weight of the digested rPET product decreased as time passed.

Increasing the reaction temperature resulted in lower molecular weight products. Although additional time was needed to raise the temperature of the reaction mixture, the additional time needed was 3 h. The additional time was most likely not sufficient alone to be a factor in the observed results.

### Mixed Green and Clear rPET Digestions

Digestion experiments were performed with a mixture of green and clear rPET to determine the effect of blending these recycled materials, i.e., to determine whether the separation of green and clear rPET is necessary for the digestion reactions to occur. Sample Ref. No. 733-18 is summarized in Table 20; 1:1 mixture of green and clear PET was digested with ethylene glycol and zinc acetate using stoichiometry which produced material used for most of the predigestion experiments. The reaction behaved normally. GPC showed that the resulting material was a digested rPET having a MP=701 and polydispersity of PI=1.112.

TABLE 20

| | ethylene | green | clear | | rxn |
|---|---|---|---|---|---|
| Ref. | glycol | rPET | rPET | Zn(OAc)2 | time |
| 733-18 | 250 mL | 155 g | 155 g | 5.00 g | 93 min |

The product from above was then used as a predigested rPET starting material in the digestion of 1:1 mixtures of green and clear rPET, and the experiments are summarized in Table 21. GPC results showed that the resulting digested rPET material had a very low polydispersity despite the fact that the clear and green rPET have different average molecular weights.

TABLE 21

| | | % predigested | | |
|---|---|---|---|---|
| | | PET (Ref. No. | | |
| Ref. | Description | 733-18) | M_{P} | PI |
| 733-19 | 155 g green PET + 155 g | 15.0% | 3546 | 1.547 |
| | clear PET + 733-18 | | | |
| 733-20 | 155 g green PET + 155 g | 25.0% | 2380 | 1.421 |
| | clear PET + 733-18 | | | |
| 733-22 | 155 g green PET + 155 g | 55.0% | 1120 | 1.228 |
| | clear PET + 733-18 | | | |

It has been shown, therefore, that recycled PET (rPET) can be converted to lower molecular weight polymers by glycolysis with ethylene glycol and zinc acetate. Material which was digested in this manner can thereby become a digestion agent to produce a wide range of molecular weight polymers from rPET starting material. Low concentrations of predigested rPET used as the “starter” gave high molecular weight products while, conversely, high concentrations of predigested rPET used as the “starter” gave low molecular weight products. Experiments with green colored rPET and clear colored rPET behaved similarly to experiments where green and clear colored rPET where mixed together.

The precipitates from each of the reactions, i.e., the digested rPET oligomers, were then each separately tested as to suitability as a resin replacement and/or resin extender.

### Removal of Green Tint Remaining in Digested Green rPET

Initially, pre-digested green PET was reacted with 2,2-dimethyl-1,3-propane diol to make higher molecular weight polymers. In particular, 250 mL of toluene were brought to reflux in a 4-neck reactor fitted with a Dean-Stark trap. 125.0 grams of pre-digested green rPET from Sample No. 732-34 (Table 22 below) were added in portions to the refluxing toluene. It appeared that the refluxing toluene could accept more than the 125.0 grams of digested rPET material.

The homogeneous green reaction mixture was heated at reflux (111.0-113.1° C.) for 6 hours after completing the digested rPET addition. 14.6 mL of water was collected from the Dean-Stark trap. As the reaction mixture cooled through 102.7° C., a 2-phase mixture formed on stopping the agitator, an upper, homogeneous green layer and a lower, opaque faint green layer. At approximately 75° C. the mixture began to solidify. After cooling to ambient temperature, the heterogeneous mixture was transferred to a beaker and allowed to stand at ambient temperature overnight. Decanted homogeneous green solution was used to obtain complete transfer to the beaker.

Vacuum filtration of the mixture produced a white filter cake having a faint blue-green tint and a lime green homogeneous filtrate. The filter cake was washed with 50 mL of toluene. The filter cake yielded 105.26 grams of white solid material after drying the solid on a 40-50° C., Buchi pump rotary evaporator for 15 minutes followed by 15 minutes on a 40° C., 0.1 mm Hg vacuum Kugelrohr. 0.32 grams of green semi-solid were recovered from the concentration of 50 mL of the green filtrate on a rotary evaporator at 40-50° C. and Buchi pump vacuum followed by 15 minutes on a 40° C., 0.4 mm Hg vacuum Kugelrohr.

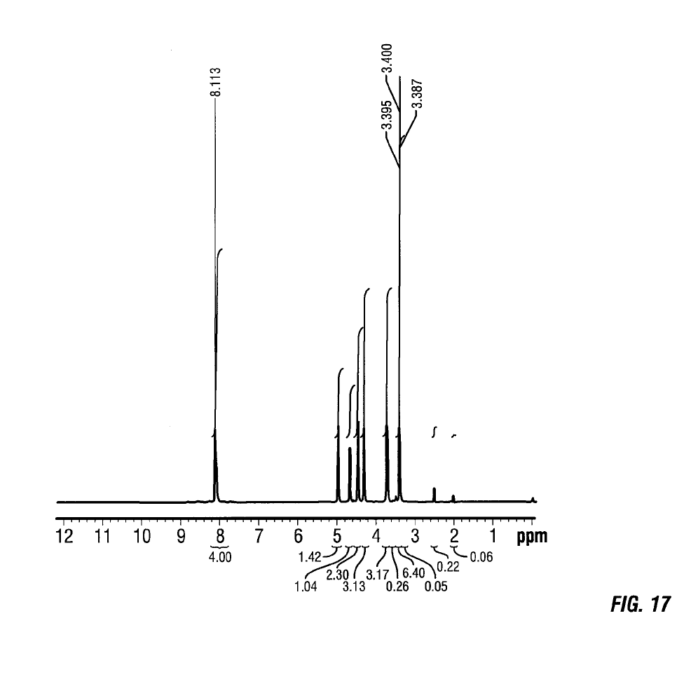

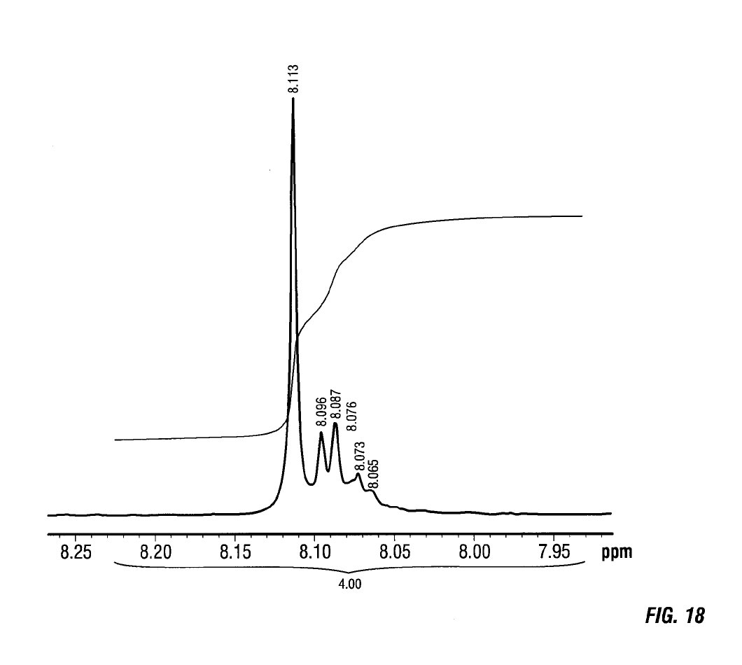

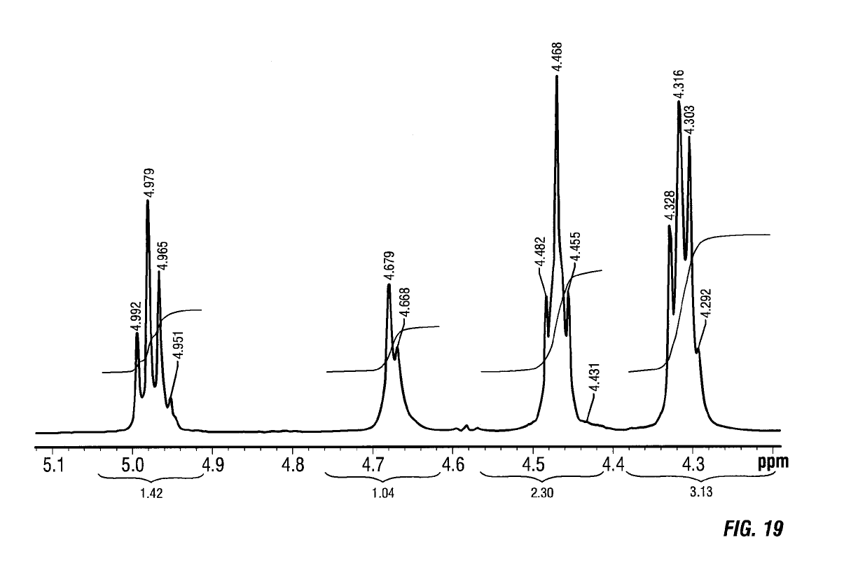

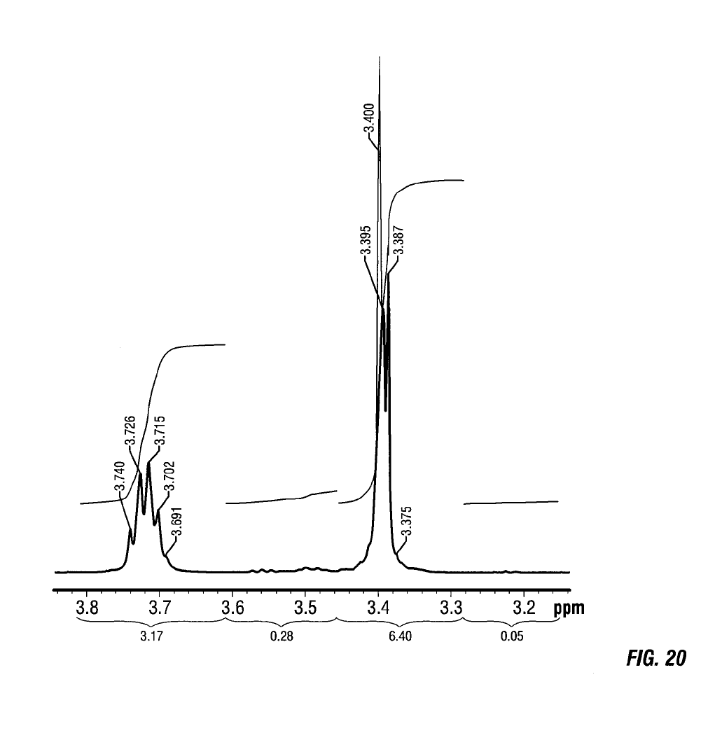

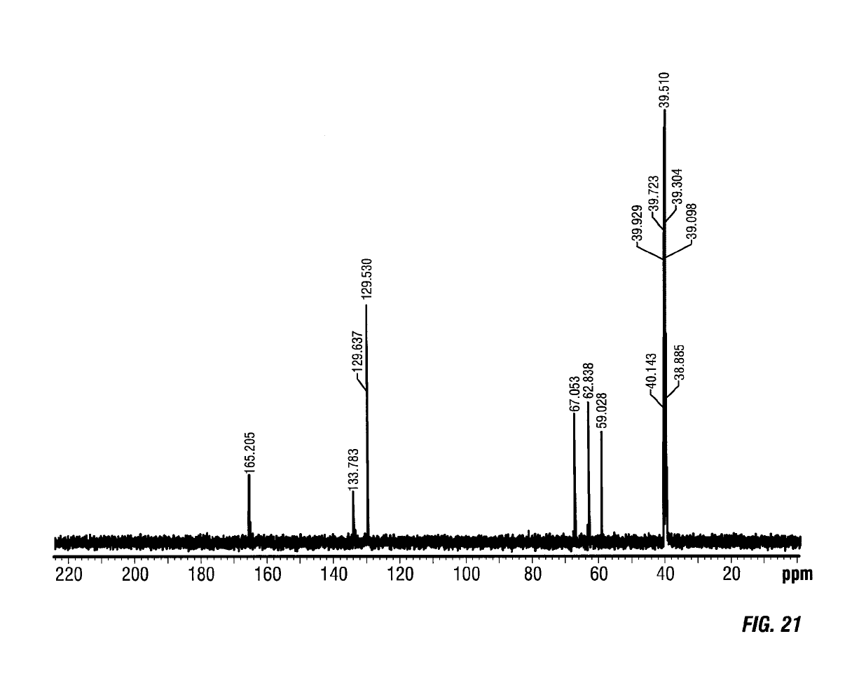

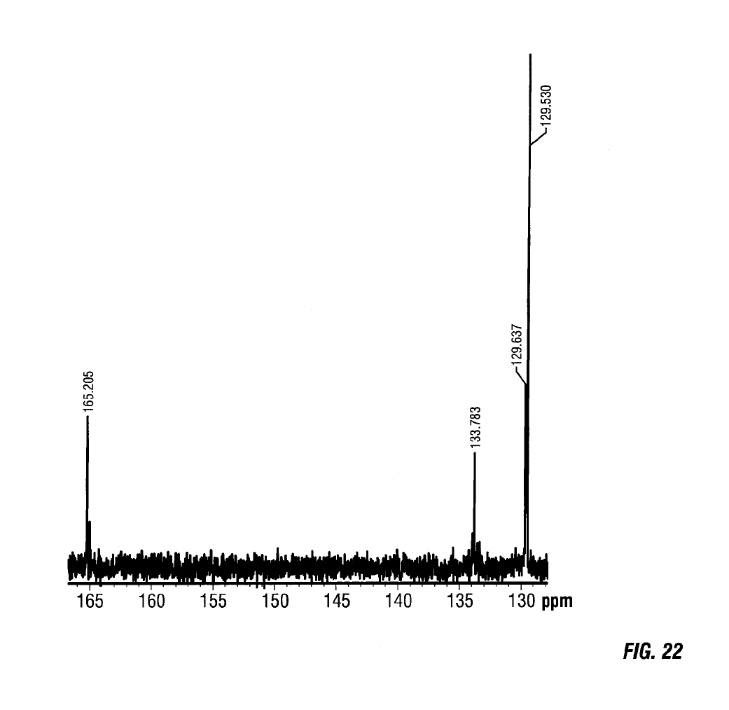

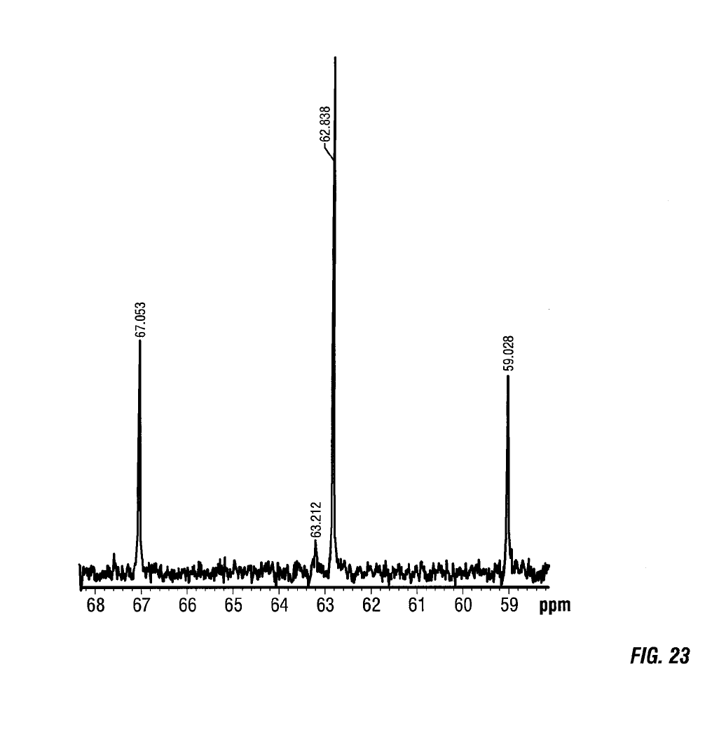

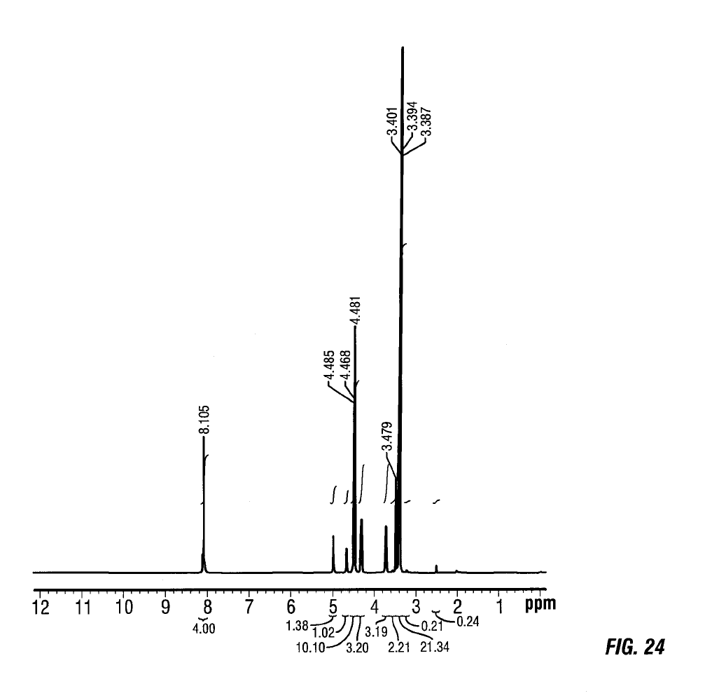

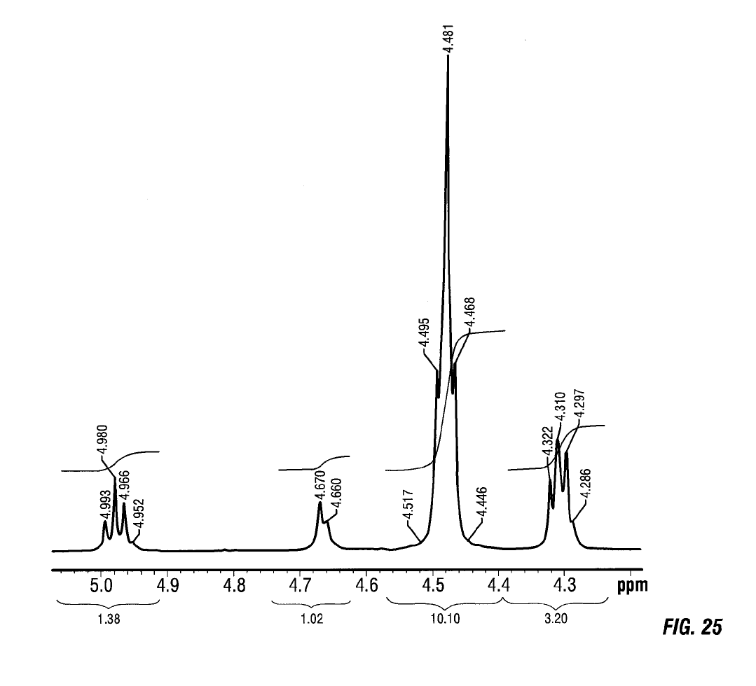

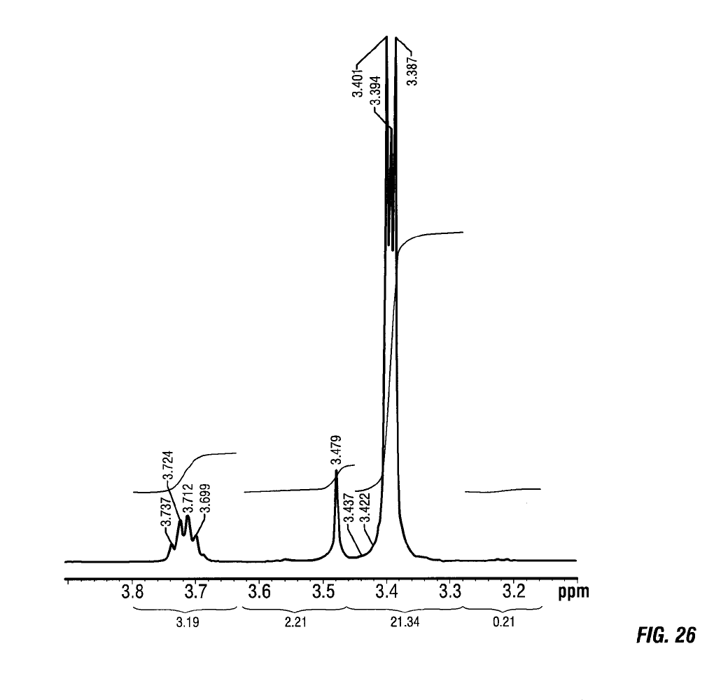

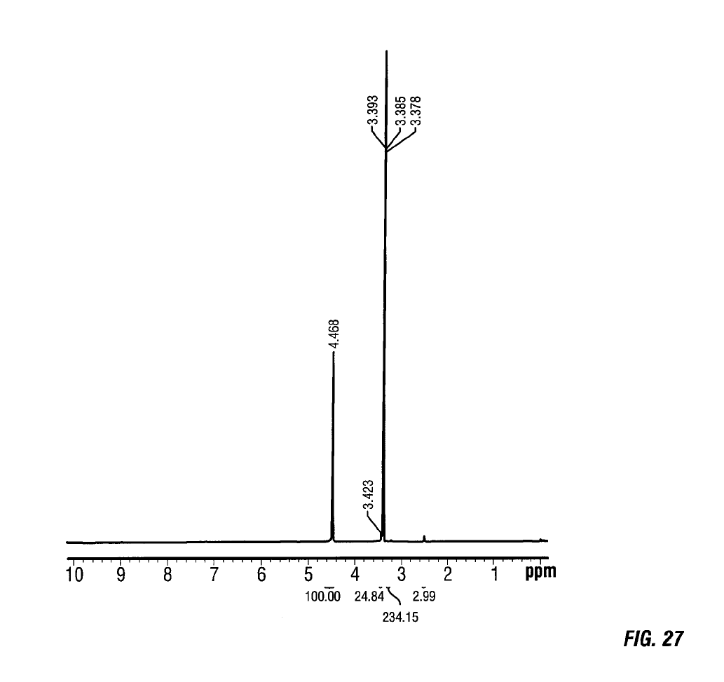

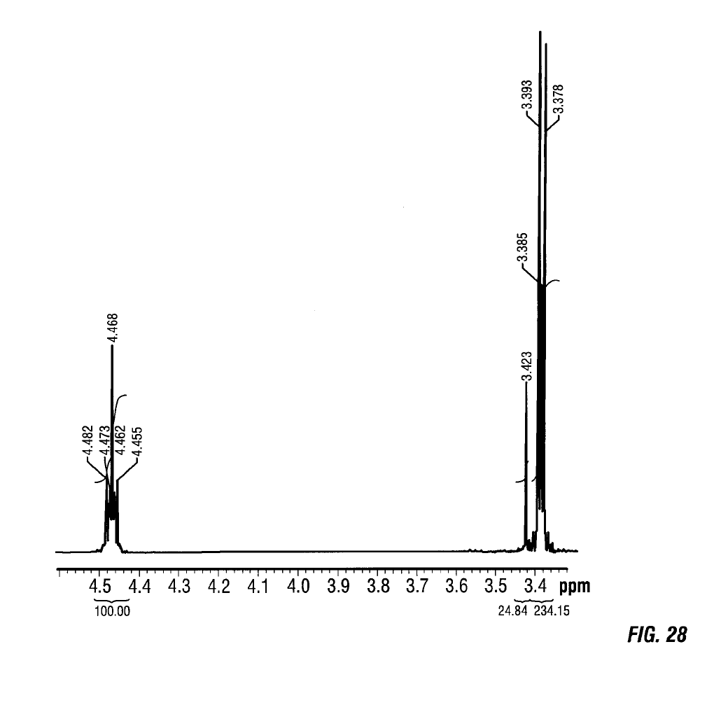

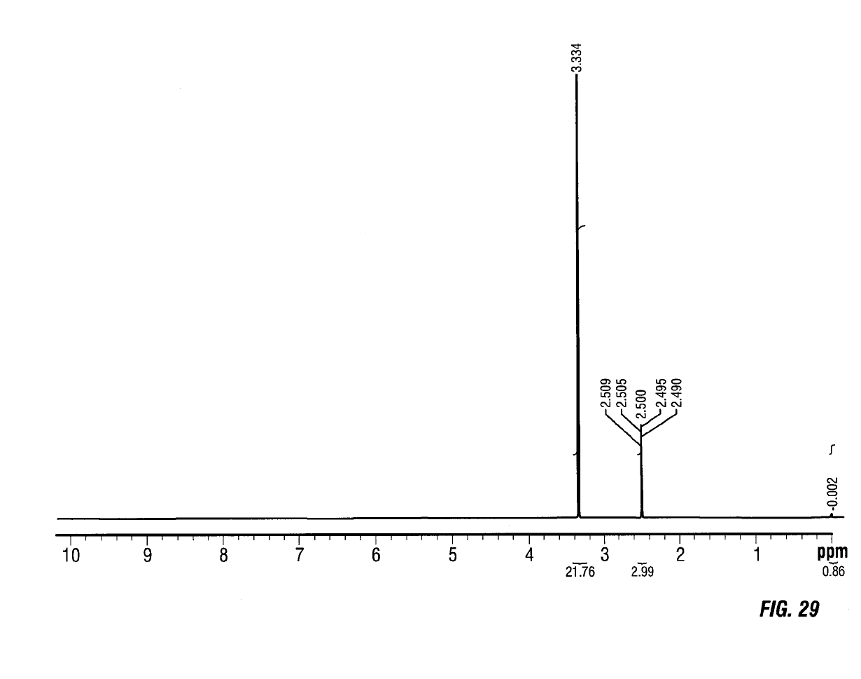

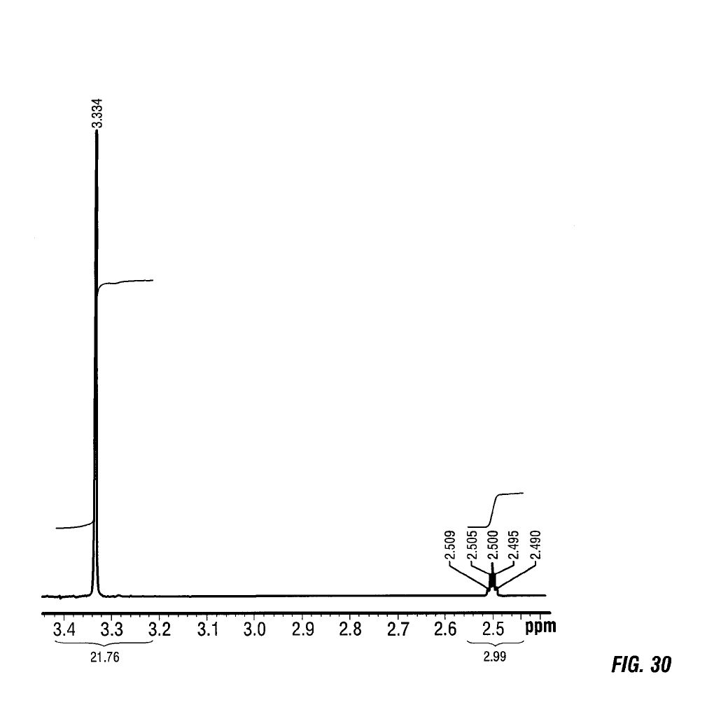

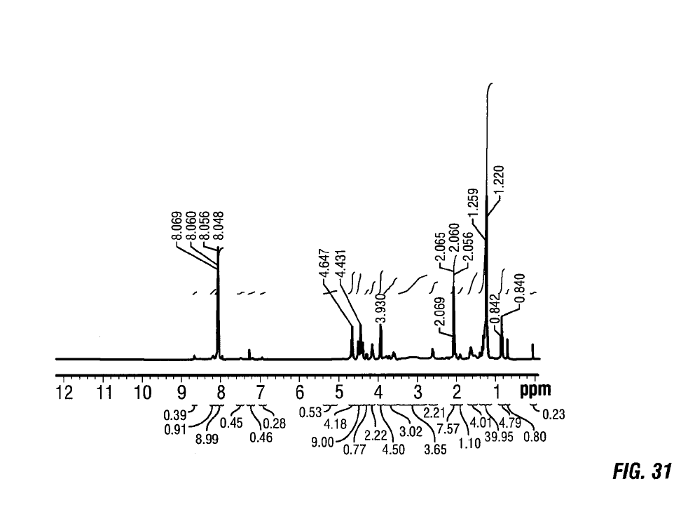

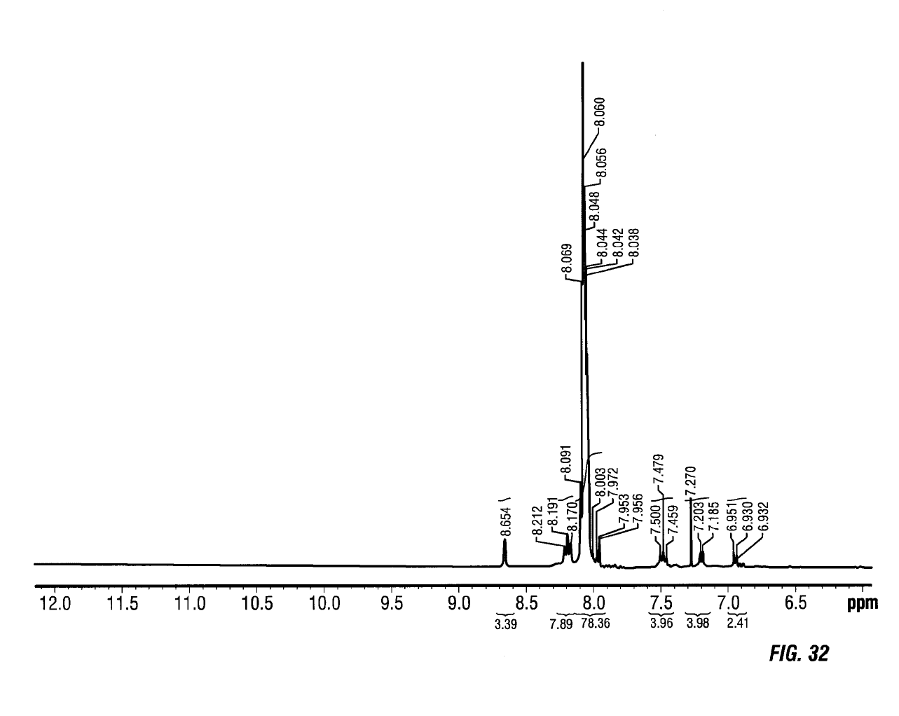

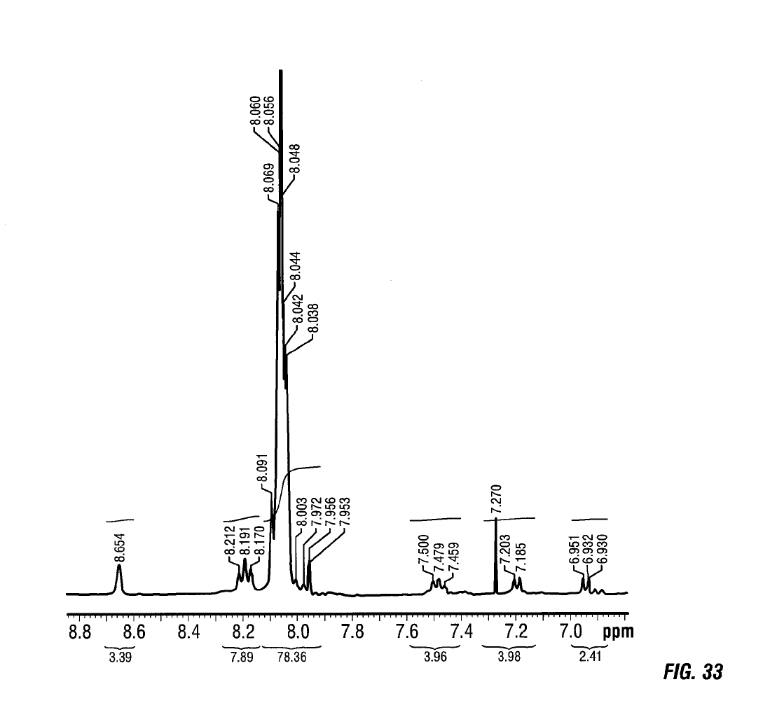

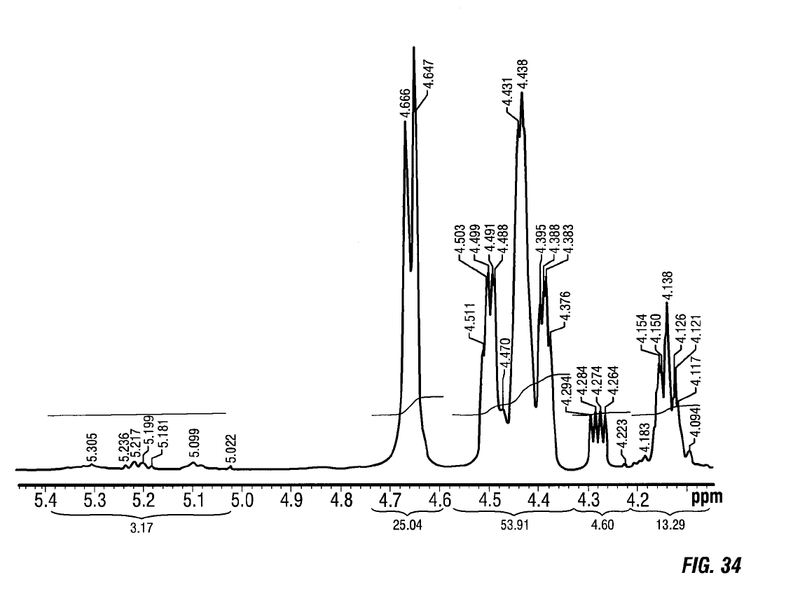

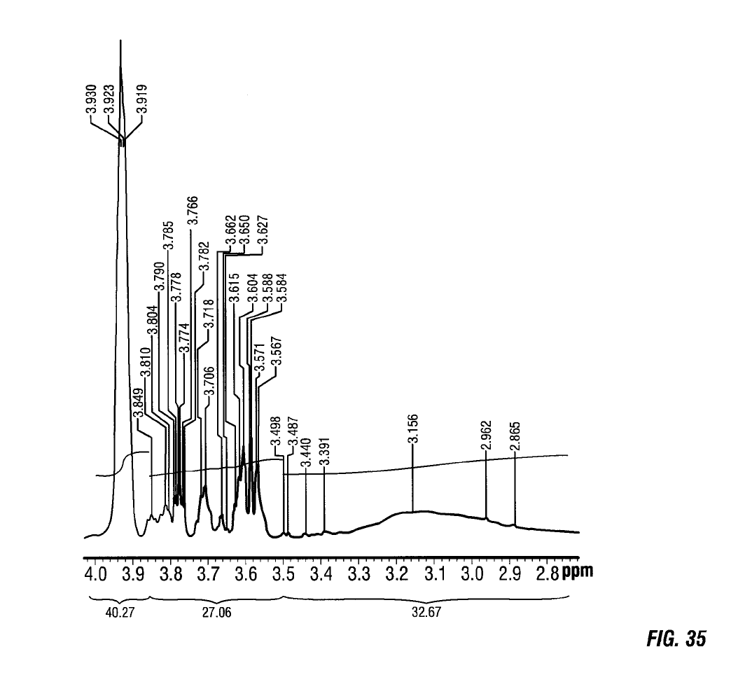

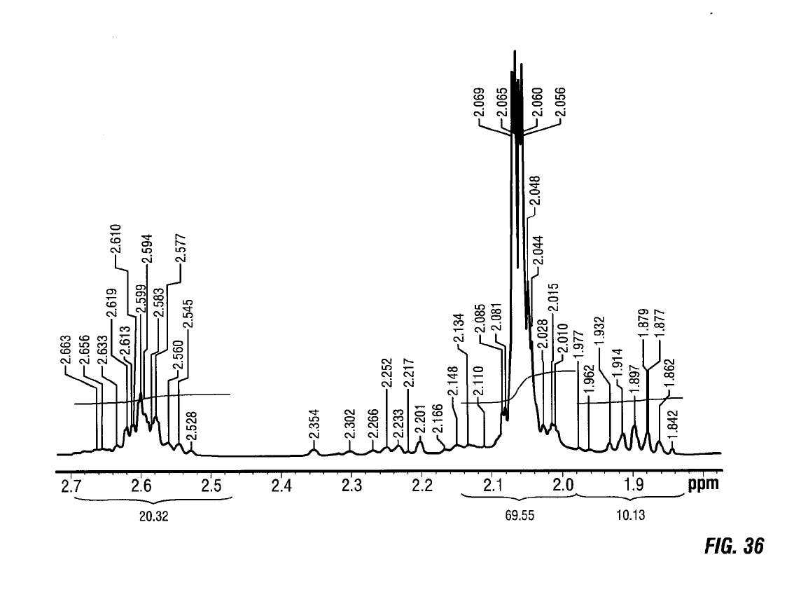

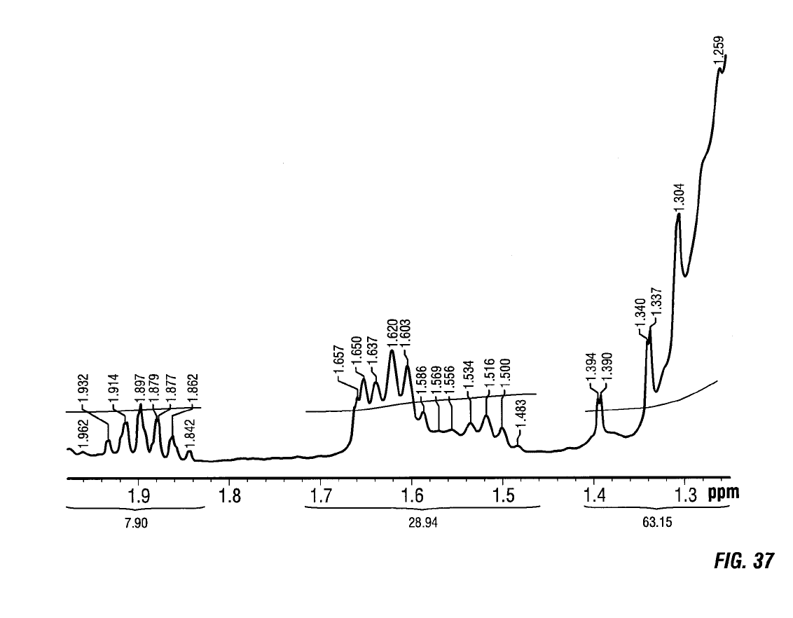

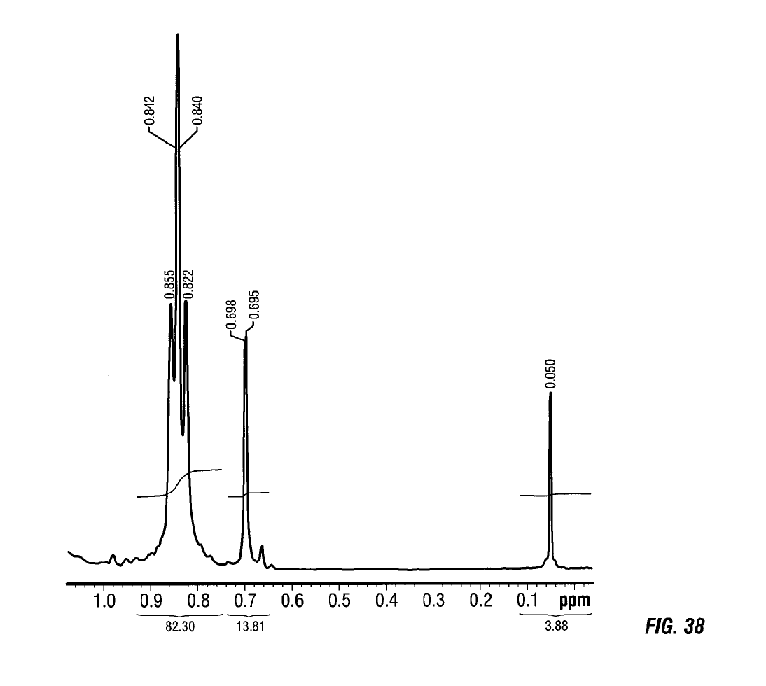

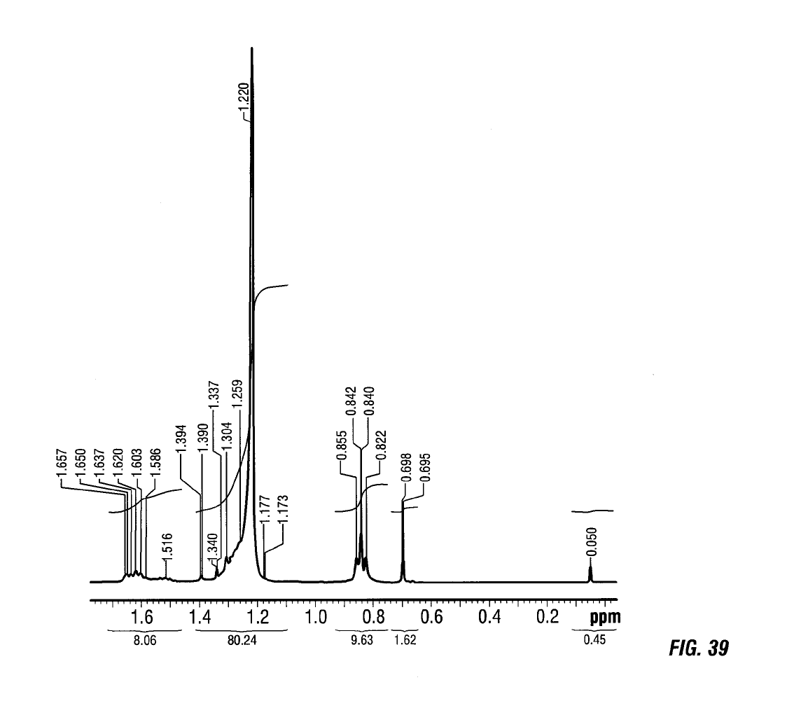

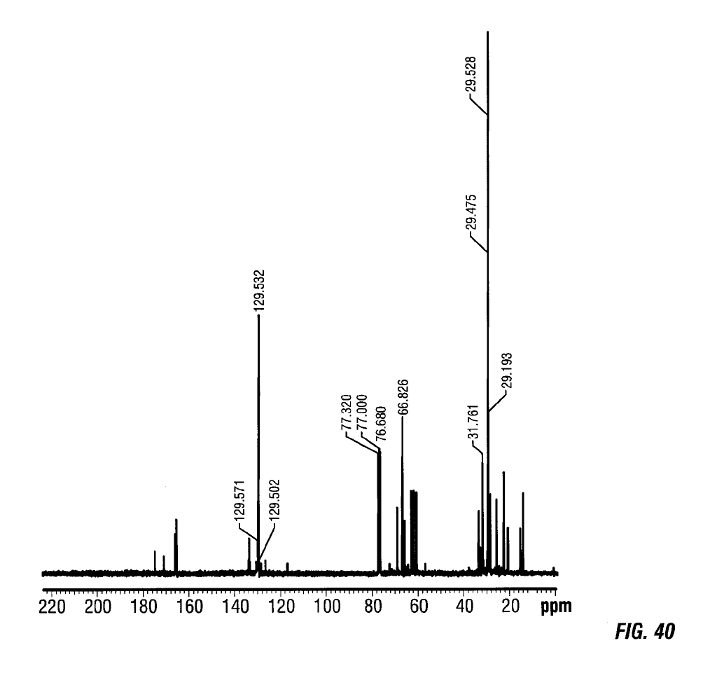

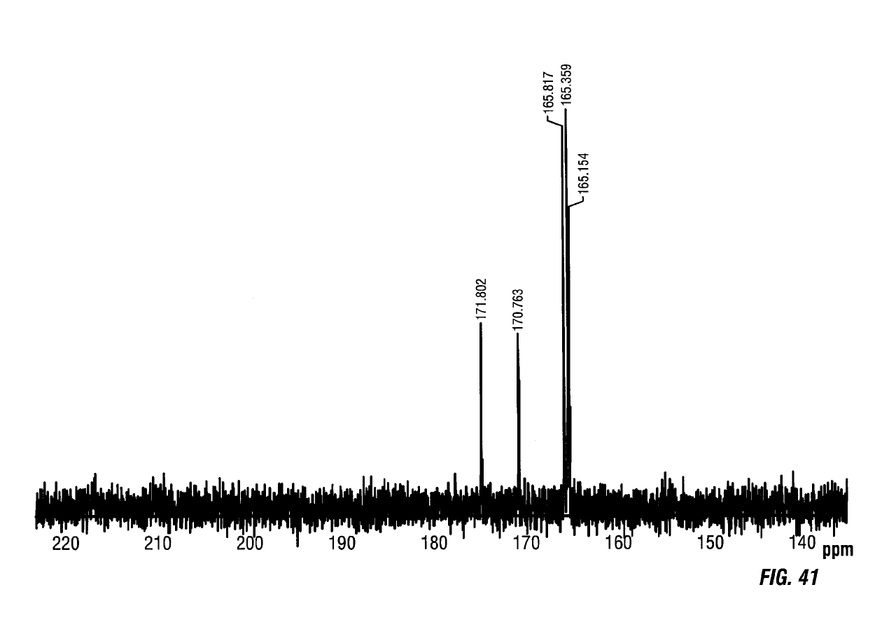

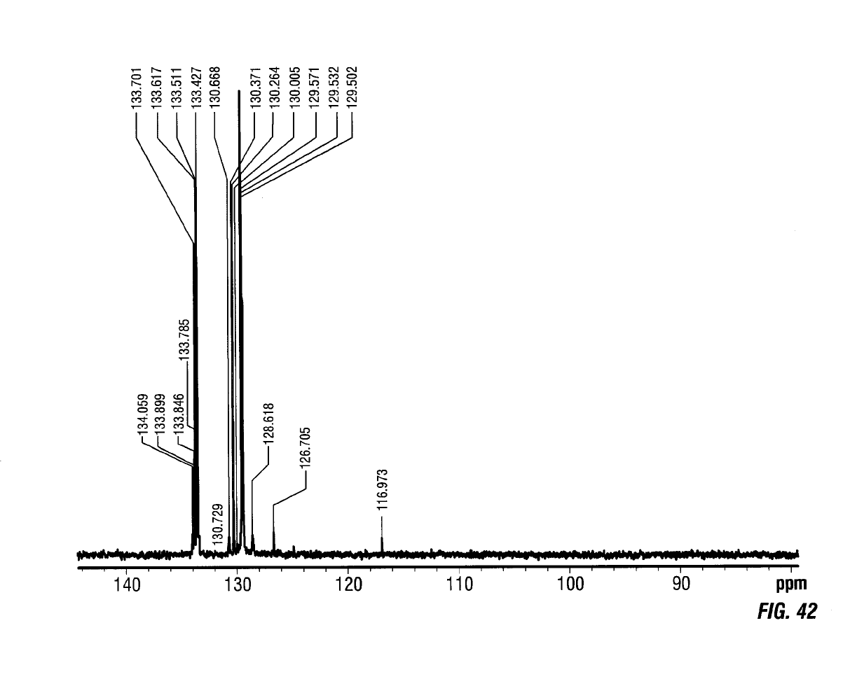

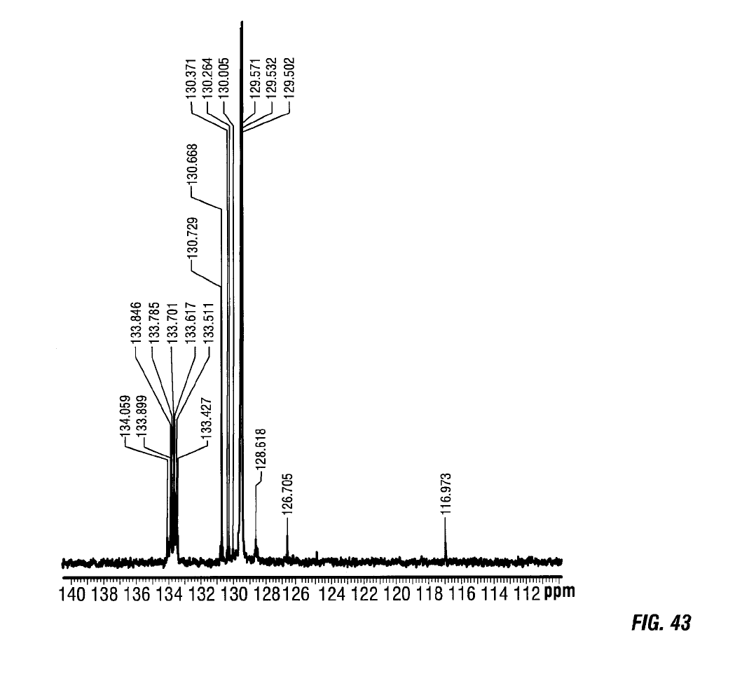

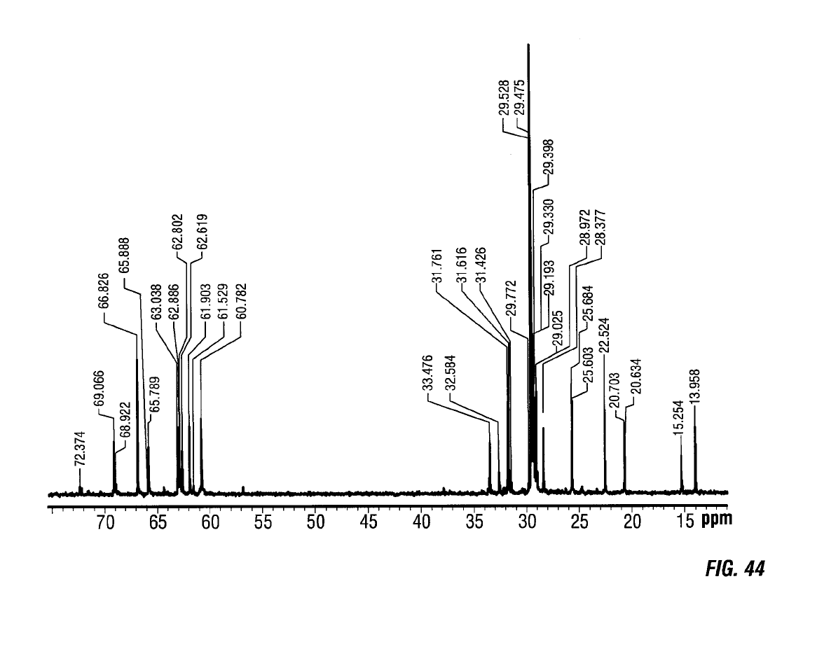

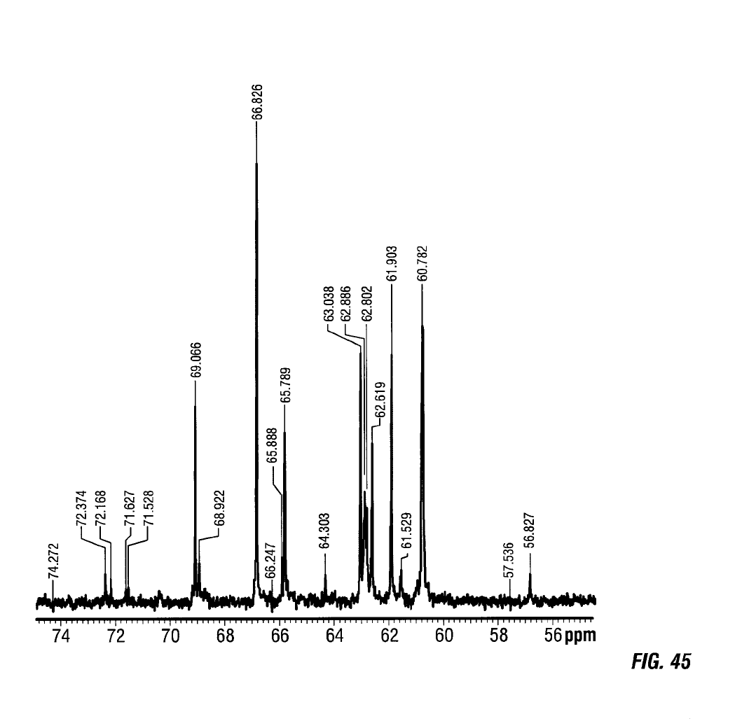

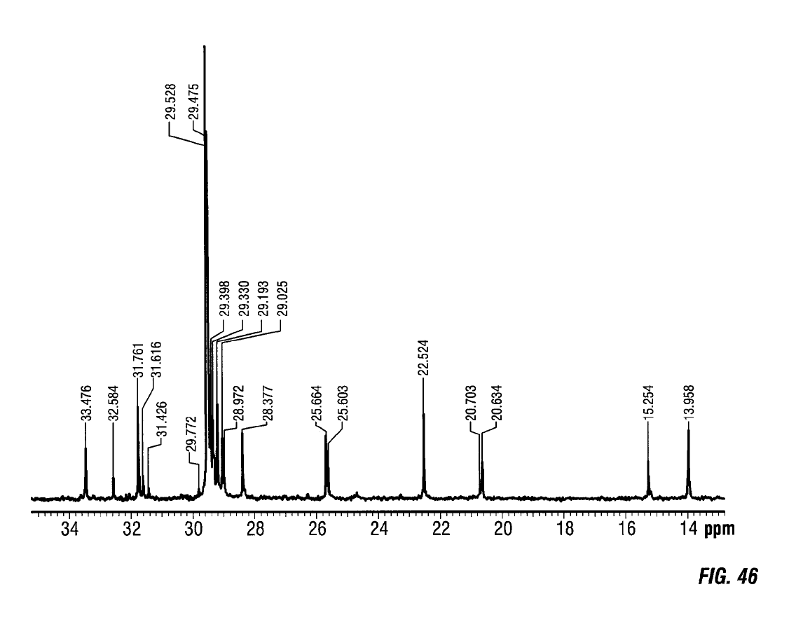

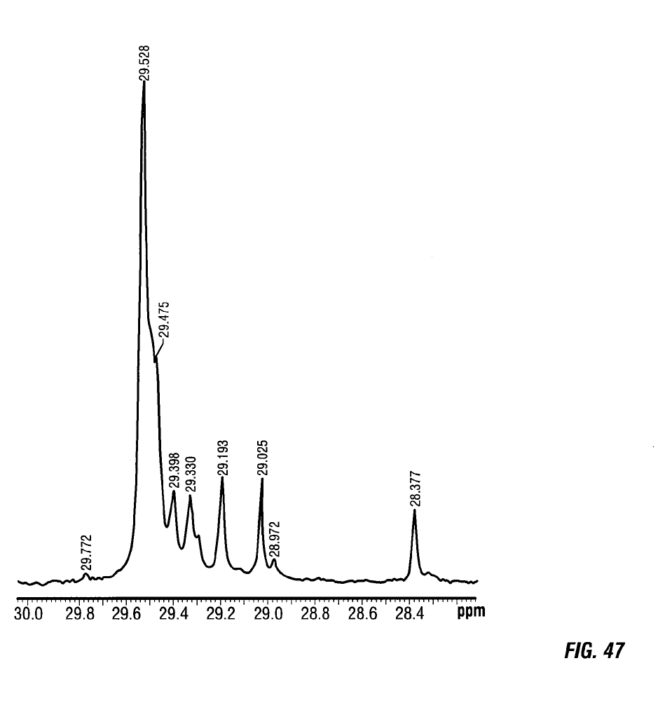

The solubility of the isolated white solid was performed with the following analysis:
Solubility of the isolated white solid
   Mostly soluble: DMSO
   Somewhat soluble: chloroform; methylene chloride
   Insoluble: acetone; acetonitrile; methanol
¹H and ¹³C NMR spectra of the white solid and isolated green filtrate solid; (¹H NMR only of white solid spiked with ethylene glycol, ethylene glycol and DMSO-d6 blank) were obtained and are shown in FIGS. 17-47. White solid melting point: first signs of melting at 81° C.; clear material at 135° C.

Thus, it has been shown that any remaining green tint in the digested rPET can be substantially removed without affecting the properties of the digested rPET.

### dPET Solubility in Organic Solvents

The digestion of recycled PET into discrete molecular weight ranges was developed at Chemir Analytical Services. These materials were all solids and the general trend was higher average molecular weight material was harder than lower average molecular weight material.

All of the materials were very insoluble in common organic solvents as well as water. For instance, the average molecular weights were determined by GPC analyses and sample preparation involved soaking the solids in dichloroacetic acid and chloroform for 1+hours until it dissolved.

In preparation for exploring commercial uses of rPET, it was necessary to develop a form of dPET which was readily soluble in common organic solvents. Among the ways to do this include:
1. Use of alcohols other than ethylene glycol;
2. Digestion with amines, diamines and polyamines;
3. Combination of ethylene glycol and 2;
4. Reaction of dPET with carboxylic acids or anhydrides to append the hydroxyl end groups.

### Green rPET Treated with Amines and Polyols

Green rPET was treated with various amines and polyols. The reactions were run as previously described: mixtures of green rPET pellets and the amine and/or polyol were heated with zinc acetate. Some reactions included ethylene glycol whiles others did not.

Diamines and hexamethylenetetramines produced hard brittle solids. This was presumably caused by cross-linking between various oligomers/low molecular weight polymers. Although these experiments did not give promising looking material, if indeed cross-linking occurred, the use of limited amounts of diamines/polyamines might prove useful in future experiments as the synthesis of higher molecular weight components from digested PET are targeted.

The reactions run with glycerin and 2,2-dimethyl-1,3-propanediol were promising. A reaction with 1.0 eq 2,2-dimethyl-1,3-propanediol (with respect to monomer MW) and green rPET gave a green pliable material, increasing to 2.0 eq of the diol gave a viscous green syrup. Several other experiments with this diol gave material which ranged from a syrup to a green brittle solid. When glycerin was used, higher amounts of glycerin gave gelatinous material. The results are summarized in Table 22.

TABLE 22

| Ref. | | Ethylene | | | | | |
|---|---|---|---|---|---|---|---|
| No. | rPET | Glycol | 2^{nd} Additive | Zn(OAc)₂ | Note | Mₚ | PI |
| 733-31 | green, | 0.50 eq | 0.19 eq 1,8- | 0.014 eq | Heated to 200- | 1707 | 1.386 |
| | 310 g | | diaminooctane | | 220 C. for 2 h. | | |
| | | | | | Opaque mint | | |
| | | | | | green brittle | | |
| | | | | | solid. | | |
| 733-32 | green, | 0.50 eq | 0.42 eq | 0.014 eq | Stirred easily | 1060 | 1.233 |
| | 310 g | | ethylenediamine | | during heat up, | | |
| | | | | | reaction | | |
| | | | | | solidified at | | |
| | | | | | 180 C. Added | | |
| | | | | | 1.0 eq EG and | | |
| | | | | | 0.42 eq | | |
| | | | | | ethylenediamine | | |
| | | | | | and temperature | | |
| | | | | | increased to | | |
| | | | | | 200 C. Solid | | |
| | | | | | remained after | | |
| | | | | | 2 h additional | | |
| | | | | | heating. | | |
| | | | | | Presumably, | | |
| | | | | | cross-linking | | |
| | | | | | occurred. | | |
| 733-35 | green, | 0.50 eq | 0.27 eq | 0.014 eq | Heated at 200 C. | 1015 | 2.602 |
| | 310 g | | hexamethylenetet | | for 52 min to give | | |
| | | | raamine | | tar like solution. | | |
| | | | | | Solidified to a | | |
| | | | | | dark, hard solid. | | |
| 733-33 | green, | 0.50 eq | 0.33 eq glycerin | 0.014 eq | Heated at 200- | 1007 | 1.262 |
| | 310 g | | | | 210 C. for 79 min | | |
| | | | | | to form solution. | | |
| | | | | | Mint green wax- | | |
| | | | | | like solid. | | |
| 733-34 | green, | 0.50 eq | 0.24 eq 1,6- | 0.014 eq | Heated to 200 C. | 1301 | 1.303 |
| | 310 g | | hexanediol | | for 178 min for a | | |
| | | | | | solution to form. | | |
| | | | | | Mint green | | |
| | | | | | opaque taffy | | |
| 733-36 | green, | 2.79 eq | — | 0.014 eq | Green rPET | 672 | 1.113 |
| | 310 g | | | | digestion with | | |
| | | | | | new rPET lot, | | |
| | | | | | standard | | |
| | | | | | stoichiometry. | | |
| | | | | | Performed to | | |
| | | | | | demonstrate | | |
| | | | | | technique is | | |
| | | | | | reproducible. | | |
| 733-37 | green, | — | 1.0 eq glycerin | 0.014 eq | Heated to 200- | 1034 | 1.242 |
| | 310 g | | | | 225 C. got 3 h. | | |
| | | | | | Material | | |
| | | | | | resembled lime | | |
| | | | | | green jello. | | |
| 733-38 | green, | — | 0.75 eq glycerin | 0.014 eq | Heated to 200 C. | 1030 | 1.303 |
| | 310 g | | | | for 67 min. | | |
| | | | | | Material | | |
| | | | | | resembled lime | | |
| | | | | | green jello. | | |
| 732-27 | green, | 0.50 eq | 0.3 eq 2,2- | 0.014 eq | Heated at 210 C. | 1132 | 1.242 |
| | 310 g | | dimethyl-1,3- | | for 2 h. Opaque | | |
| | | | propanediol | | green pliable | | |
| | | | | | material, | | |
| | | | | | resembled | | |
| | | | | | Gumby. | | |
| 732-28 | green, | — | 0.6 eq 2,2- | 0.014 eq | Heated at 220 C. | 1563 | 1.297 |
| | 310 g | | dimethyl-1,3- | | for 5.5 h. Clear | | |
| | | | propanediol | | green sticky | | |
| | | | | | pliable material, | | |
| | | | | | will take and hold | | |
| | | | | | shape when | | |
| | | | | | handled. | | |
| 732-29 | green, | — | 1.0 eq 2,2- | 0.014 eq | Heated at 220 C. | 1224 | 1.226 |
| | 310 g | | dimethyl-1,3- | | for 2.5 h. Steady | | |
| | | | propanediol | | reflux last 78 min | | |
| | | | | | (ethylene glycol?). | | |
| | | | | | Clear green sticky | | |
| | | | | | pliable material, | | |
| | | | | | will take and hold | | |
| | | | | | shape when | | |
| | | | | | handled (resembled | | |
| | | | | | 732-28). | | |
| 732-30 | green, | — | 2.0 eq 2,2- | 0.014 | Heated to 220 C. | 1142 | 1.140 |
| | 155 g | | dimethyl-1,3- | | for 3.25 h, milky | | |
| | | | propanediol | | reaction mixture, | | |
| | | | | | steady reflux. | | |
| | | | | | Opaque green | | |
| | | | | | liquid, slightly | | |
| | | | | | less viscous than | | |
| | | | | | honey. Glassware | | |
| | | | | | easily cleaned | | |
| | | | | | with acetone. | | |
| 732-31 | green, | — | 0.2 eq 2,2- | 0.014 | Heated to 220 C. | 3601 | 1.612 |
| | 310 g | | dimethyl-1,3- | | for 4.5 h, isolated | | |
| | | | propanediol | | a dark green | | |
| | | | | | brittle solid. | | |
| 732-34 | green, | 2.79 eq | — | 0.014 | Heated to 200 C. | 691 | 1.104 |
| | 620 g | | | | for 1 h to give a | | |
| | | | | | waxy green solid. | | |
| 733-46 | green, | — | 1.5 eq glycerin | 0.014 | Heated to 200- | 700 | 1.185 |
| | 310 g | | | | 240 C. for 112 min | | |
| | | | | | to give a sticky | | |
| | | | | | green gel. | | |
| 733-48 | green, | — | 0.5 eq 1,6- | 0.014 | Heated to 200 C. | 2066 | 1.378 |
| | 310 g | | hexanediol | | for 6.1 h. Cooled | | |
| | | | | | to give a hard | | |
| | | | | | mint green solid. | | |
| 733-49 | dPET | — | 0.5 eq 1,6- | — | dPET (MW 672) | 724 | 1.120 |
| | from | | hexanediol | | was melted at | 1584 | 1.301 |
| | 733-36 | | | | 150 C. and treated | 1143 | 2.211 |
| | | | | | with diol. Heated | 9 | 1.936 |
| | | | | | for 3 h, samples | 6647 | |
| | | | | | were drawn at | | |
| | | | | | t = 0, 1, 2, 3 h | | |
| | | | | | (column to right) | | |
| | | | | | and molecular | | |
| | | | | | weight increased. | | |
| | | | | | A flexible green | | |
| | | | | | solid was obtained | | |
| | | | | | after cooling. | | |

### Reaction of dPET with Carboxylic Acids and Anhydrides

Predigested material produced in experiment 733-36 from the customary procedure of 2.79 eq ethylene glycol, 1.0 eq rPET and 0.014 eq zinc acetate dihydrate was treated with a variety of diacids and anhydrides in an effort to produce higher molecular weight material. The reactions were performed by heating a mixture of the predigested material along with either a diacid or anhydride. A stream of nitrogen was blown through the flask in to remove water which was produced as a by-product. Oxalic acid was the smallest of the diacids used, and it gave a hard green solid. Higher molecular weight diacids and anhydrides gave materials which ranged from pliable and sticky jello-like material. GPC data is presented for different times in descending order are t=0, t=1 h, t=2 h and t=3 h (or final time point as indicated). The results are summarized in Table 23.

TABLE 23

| | Predigested | | | | | |
|---|---|---|---|---|---|---|
| Ref. No. | PET | Additive | Notes | | Mₚ | PI |
| 733-39 | 1.0 eq | 0.5 eq oxalic acid | Exothermic reaction with considerable outgassing. Heated 3 h at 150C. Isolated hard green solid. | 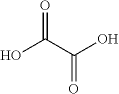 | 676 686 706 729 | 1.121 1.131 1.128 1.130 |
| 733-40 | 1.0 eq | 0.5 eq D- malic acid | Heated 3 h at 150C. to give a mint green gummy material, very sticky, viscous syrup which could not be separated from aluminum sheet. | 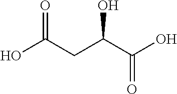 | 697 691 1025 1022 | 1.120 1.149 1.193 1.232 |
| 733-42 | 1.0 eq | 0.5 eq malonic acid | Heated 3 h at 150C. to give a mint green gummy solid. Material could be hand shaped and hold its form. Sticky, could not be separated from aluminum sheet. | 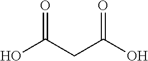 | 687 693 1038 1076 | 1.110 1.146 1.182 1.205 |
| 733-43 | 1.0 eq | 0.5 eq tartaric acid | Heated for 3 h at 150C. to give a hard green solid. | 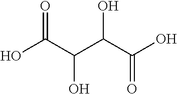 | 707 706 716 673 | 1.121 1.131 1.140 1.171 |
| 733-44 | 1.0 eq | 0.5 eq L- malic acid | Heated for 3 h at 150C. to give a pliable green solid. Sticky, pliable enough to take and hold a thumb print impression. | 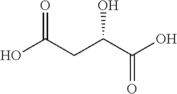 | 659 645 997 1004 | 1.113 1.150 1.191 1.212 |
| 733-45 | 1.0 eq | 0.5 eq maleic anhydride | Heated for 3 h at 150C. to give a clear green sticky jello-like solid. | 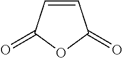 | 670 1006 1372 1646 | 1.107 1.327 1.366 1.432 |
| 733-47 | 1.0 eq | 0.5 eq succinic anhydride | Heated for 3 h at 150C. to give a dark brittle rubber like solid. | 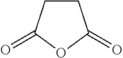 | 680 4336 16165 22674 | 1.122 1.606 2.403 3.575 |
| 733-51 | 1.0 eq | 0.5 eq propionic anhydride | Heated for 2.5 h at 150C, after 2 h the reaction mixture solidified. Increased temp to 250C. to give a thick syrup which cooled to a hard mint green solid. | 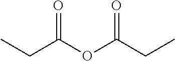 | 791 765 1861 13499 | 1.104 1.122 1.293 2.222 |
| 733-52 | 1.0 eq | 0.5 eq methacrylic anhydride | Heated for 2.5 h at 150C. A hard brittle green solid was obtained. | 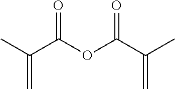 | 766 1375 2098 5248 | 1.143 1.206 1.289 5.433 |
| 733-53 | 1.0 eq | 0.5 eq glutaric anhydride | Heated for 3 h at 150C. Upon cooling, a gummy mint green mass formed. The material could be deformed and hold a shape. | 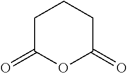 | 828 1230 1242 1593 | 1.096 1.167 1.204 1.260 |
| 733-54 | 1.0 eq | 0.5 eq glutaric acid | Heated for 3 h at 150C. Upon cooling, a mint green play- dough like mass formed. The material could be deformed and hold a shape, it was harder to manipulate than 733-53. | 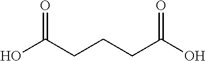 | 842 1246 1581 2366 | 1.106 1.255 1.286 1.334 |
| 733-55 | 1.0 eq | 0.5 eq D- malic acid | dPET was stirred in refluxing toluene and treated with D- malic acid. Refluxed for 3 h with a Dean- Stark trap to collect water. Cooled and collected a gummy off-white solid. (This experiment was similar to 733-40 but with toluene, the previous experiment could not be separated from aluminum foil used in collecting it.) | 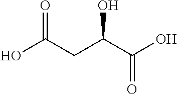 | 829 | 1.107 |
| 733-56 | 1.0 eq | 0.5 eq maleic anhydride | dPET was stirred in refluxing toluene and treated with maleic anhydride. Refluxed for 3 h with a Dean- Stark trap to collect water. Cooled and collected a gummy off-white solid. | 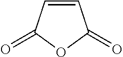 | 852 | 1.117 |
| 733-61 | 1.0 eq | 0.25 eq trimellitic anhydride | dPET was melted at 150C. and treated with trimellitic anhydride. This was selected to provide cross- linking between oligomers. A sampled at t = 0 when the anhydride was added and at 20 min when the reaction mixture solidified. | 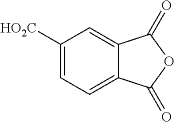 | t = 0: 677 t = 20 min: 2339 | 1.122 2.544 |
| 733-62 | 1.0 eq | 0.25 eq trimellitic anhydride | dPET (732-3, clear MW 672) was stirred in refluxing toluene and treated with trimellitic anhydride for 3 h. Reaction was similar to 733-61 but with solvent to facilitate mixing and removal from reaction flask. Upon cooling to r.t., a hard white solid separated from the toluene layer. | 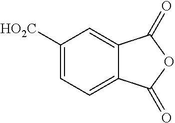 | 701 | 1.117 |
| 733-64 | 1.0 eq | 0.1 pyromellitic anhydride | dPET (732-3, clear MW 672) was stirred in refluxing toluene and treated with pyromellitic anhydride for 3 h. Upon cooling to r.t., a hard white solid separated from the toluene layer. It had the same texture as st.material. | 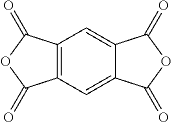 | 714 | 1.139 |

### Solubility Studies

Several of the resulting dPETs were taken up in toluene, NMP, THF and water. dPET from 732-28, 732-29 and 732-30 showed excellent solubility in NMP.

### Toner Formulation Using rPET

In order to test the rPET oligomers (i.e., the digested rPET materials) produced according to the exemplary reactions of Table 3 as a resin extender or resin replacement compound, the rPET oligomers were formulated into a toner composition for use in a laser printer. Although a toner composition is disclosed, it should not be considered as limiting. The rPET oligomers disclosed herein can be used generally as resin extenders and/or resin replacement compositions for a wide range of products. For example, paints, coatings, adhesives, personal care, health and beauty, plastics, fibers, textiles, etc. One of ordinary skill in the art, given the present specification, would appreciate the different and varied uses to which the rPET oligomers can be put.

The toner composition formulation used to test the rPET oligomers was based on a deformulation process conducted on a toner composition having the trade name IPQ-2® produced by Canon, i.e., a standard and commonly accepted toner composition in general commercial use. Through deformulation, it was determined that the IPQ-2® product was comprised generally of a binder (for example, propoxylated Bisphenol A epoxy and polyester resin) and additives (for example, carbon black, silica and other additives).

In addition to the rPET oligomer product, a PET compound (bis(2-hydroxyethyl)terephthalate)) manufactured by Aldrich was also studied. Although the Aldrich PET compound is virgin material (i.e, it is not formed from a scrap or recycled PET material), the Aldrich PET compound is a suitable substitute for testing the appropriateness and useability of an additional form of PET oligomer.

TABLE 24

| Propoxylated | | | | |
|---|---|---|---|---|
| Bisphenol A | Polyester | Carbon | Magnetite | SiO₂ (amorphous |
| Epoxy | Resin | Black | (Fe₃O₄) | and cristobalite) |
| Fine-Tone | Fine-Tone | Black Pearls | — | Cab-O-Sil |
| T-6694 | T-382-ES* | LA 67 | | TG-310 G(−) |
| Reichhold | Reichhold | Cabot | | Cabot |
| Fine-Tone | PET | Recycled | — | Cab-O-Sil |
| T-RM-12 | Oligomers | Carbon | | TG-820 F(+) |
| Reichhold | According | Black | | Cabot |
| | to presently | Powder, | | |
| | disclosed | Amorphous | | |
| | and | Carbolytic | | |
| | claimed | | | |
| | concept(s). | | | |
| Fine-Tone | — | Recycled | — | — |
| T-382-ES | | Carbon | | |
| Reichhold | | Black | | |
| | | Powder, | | |
| | | Amorphous | | |
| | | Carbolytic | | |

For testing, 18 different iterations of the rPET oligomers were compared. Some of these iterations contained no recycled content and were merely used as comparative examples. Each formulation was prepared by manual mixing of all components in unlined one gallon paint cans. All the formulas without the rPET oligomers or Aldrich PET trimer mixed into uniform compositions. The samples with the PET oligomers and the Aldrich PET compound were preground by forcing the material through a number 10 mesh sieve and then processed in the same manner as all the other compositions. All formulas were Jet Milled on a 4 inch Orb Cycle jet mill and the average particle size was determined by Horiba Wet Fran.

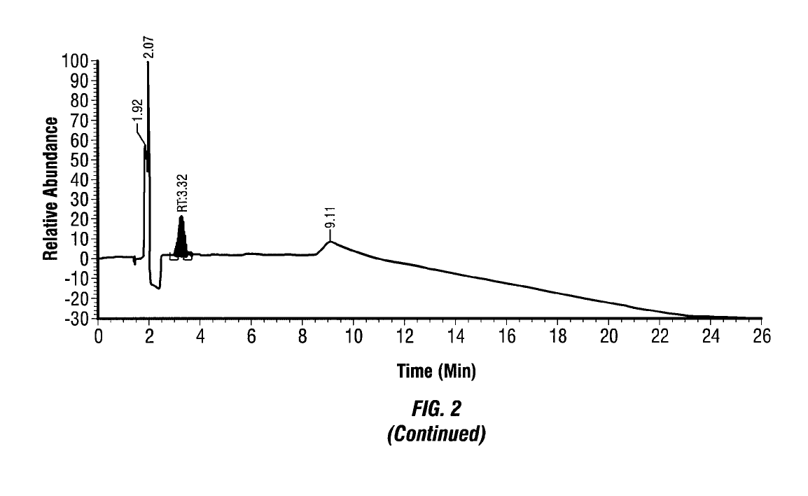

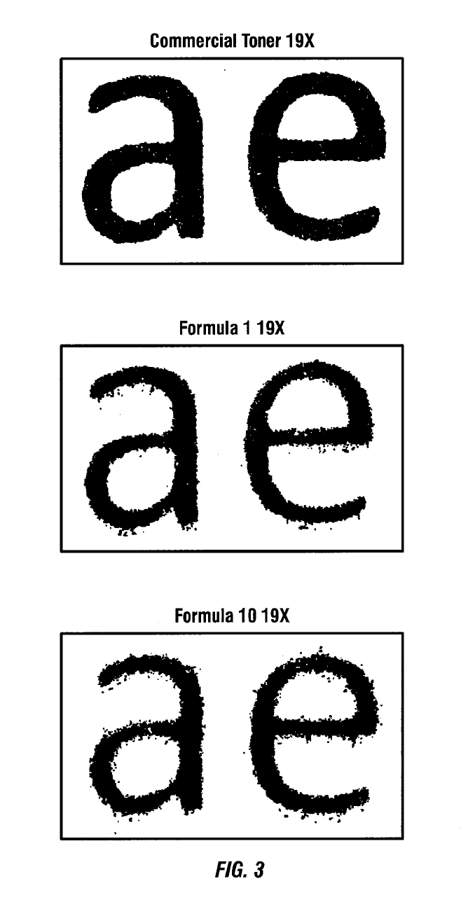

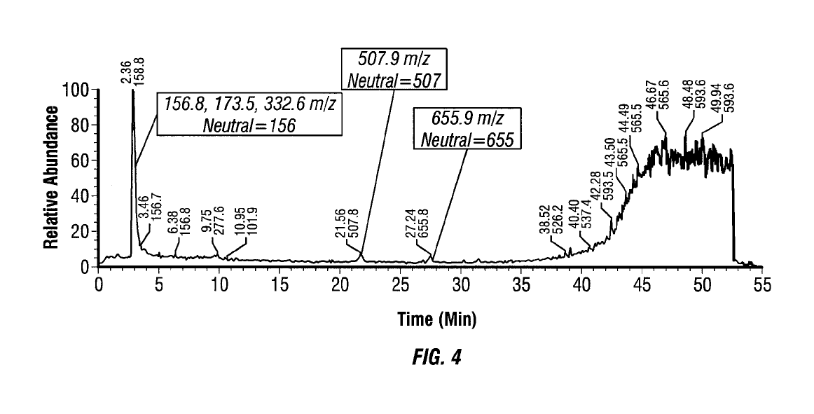

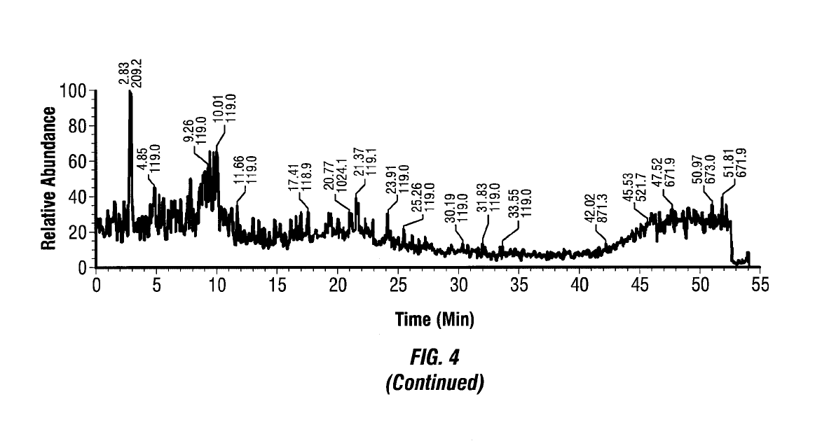

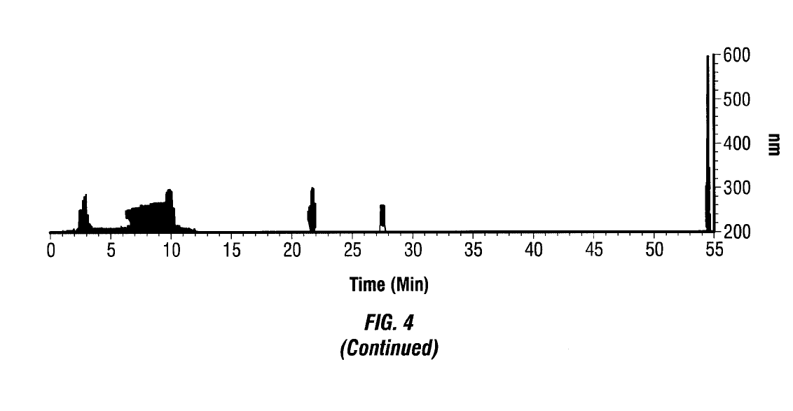

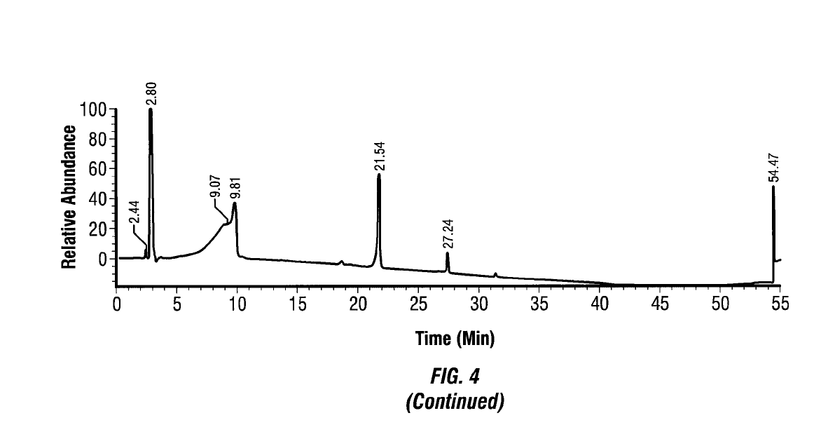

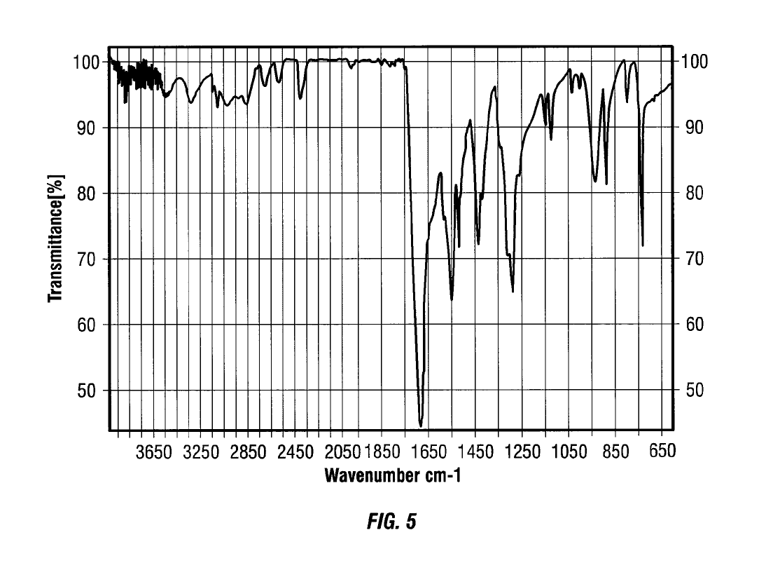

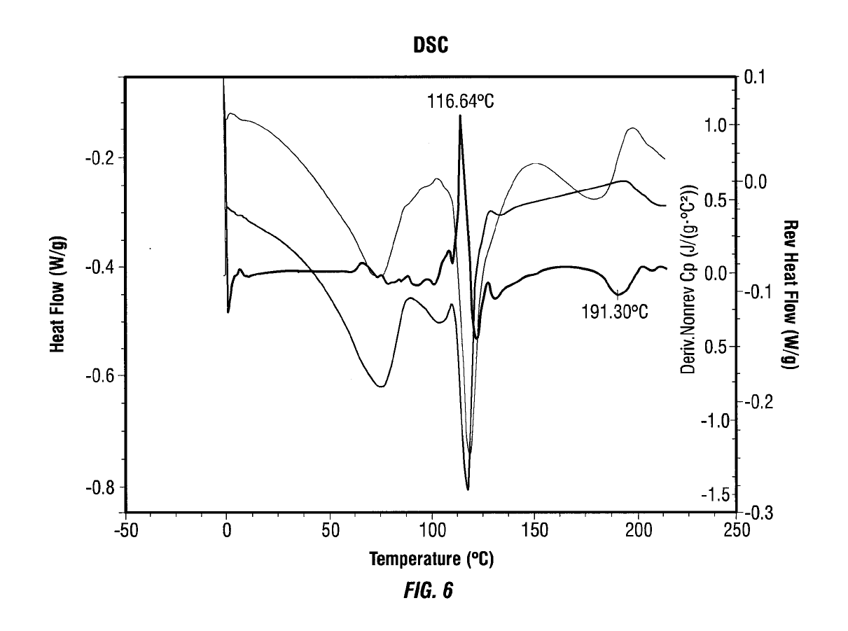

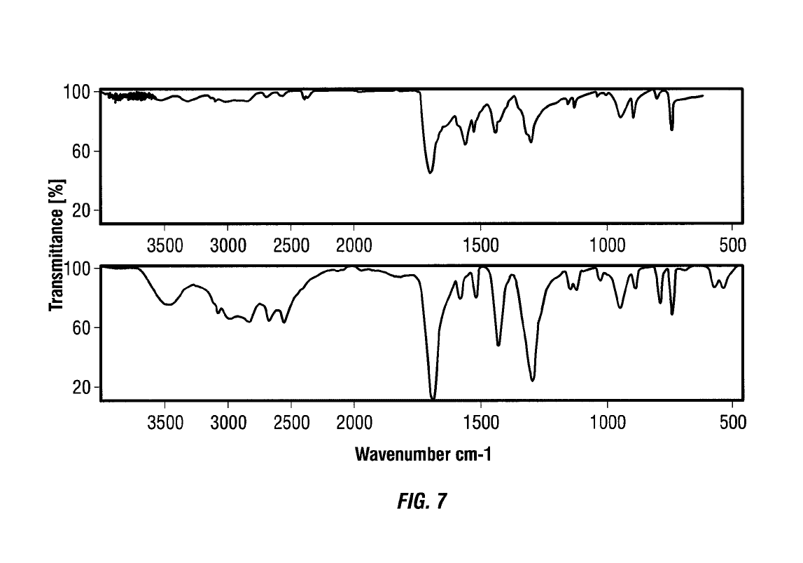

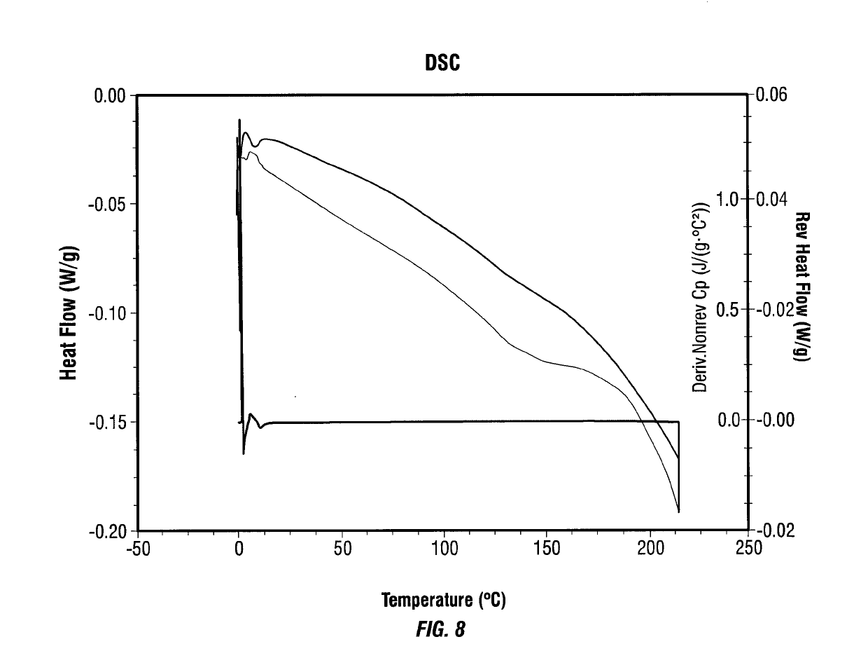

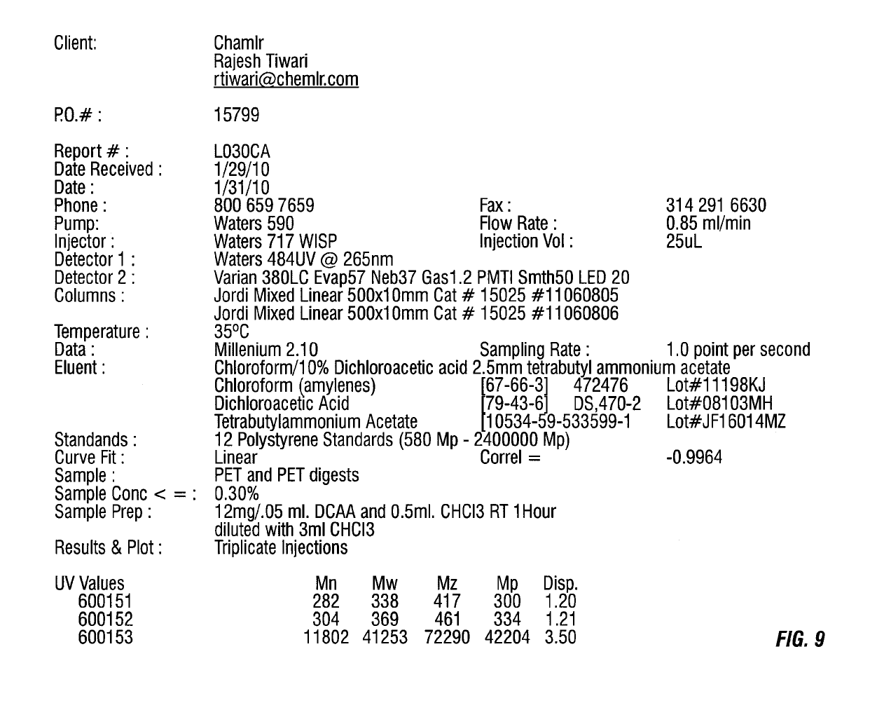

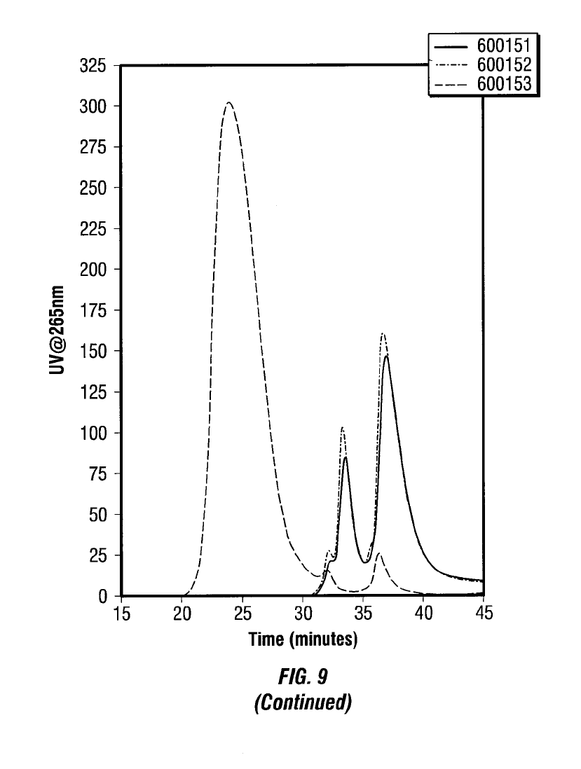

As stated, each formulation was jet milled to the proper particle size and filled into an empty 27X C4127 cartridge. The filled cartridge was used in an HP LASERJET® 4050 and printed on HAMMERMILL® 20 lb 96 brightness 8½ by 11 inch “multipurpose” paper. The first 50 sheets printed are considered flushing the cartridge of any remaining commercial toner, and then it is accepted that the formulation toner was actually printing. The sheets of paper are examined for printing and fusion to paper. FIG. 3 shows 19× magnification of printing by a Commercial Toner (i.e., the Canon IPQ-2® toner), Formula 1, and Formula 10, respectfully for comparison. A formulation of approximately 50% recycled content is acceptable because approximately 600 pages were printed with complete fusion to the paper and quality number and lettering.

TABLE 25
Formula 1-0% Recycled Content-Reformulation Baseline (−)
Composition printed and fused to the paper.

| Raw Material | Material Type | Description | Pounds |
|---|---|---|---|
| T-382-ES | Resin | Bis-A/Polyester | 385 |
| Black Pearls L A67 | Pigment | Carbon Black- virgin | 105 |
| Cab-o-SILR TG- | Charge Control | Amouphous Silica | 10 |
| 810G | Agent | (negative charge) | |
| | | | 500 |

TABLE 26
Formula 2-21% Recycled Content-Carbon Black
Composition printed and fused to the paper.

| Raw Material | Material Type | Description | Pounds |
|---|---|---|---|
| T-382-ES | Resin | Bis-A/Polyester | 385 |
| APEX CM powder | Pigment | Recycled Carbon black | 105 |
| Cab-o-SILR TG- | Charge Control | Amouphous Silica | 10 |
| 810G | Agent | (negative charge) | |
| | | | 500 |

TABLE 27
Formula 3-30% Recycled Content-PET Oligomers
Composition fused to the paper, but did not print properly. Believed
to be issue regarding not enough charge agent used.

| Raw Material | Material Type | Description | Pounds |
|---|---|---|---|
| T-6694 | Resin | Bis-A Epoxy | 240 |
| Digested | Resin | Digested Polyester | 150 |
| Polyester | | (KF) | |
| Black Pearls L A67 | Pigment | Carbon Black- virgin | 105 |
| Cab-o-SILR TG- | Charge Control | Amouphous Silica | 10 |
| 810G | Agent | (negative charge) | |
| | | | 505 |

TABLE 28
Formula 4-51% Recycled Content-PET Oligomers &
Recycled Carbon Black Composition did not grind properly
due to the PET oligomer not being dry enough. The
mill clogged and printing could not be attempted.

| Raw Material | Material Type | Description | Pounds |
|---|---|---|---|
| T-6694 | Resin | Bis-A Epoxy | 240 |
| Digested | Resin | Digested Polyester | 150 |
| Polyester | | (KF) | |
| APEX CM powder | Pigment | Recycled Carbon black | 105 |
| Cab-o-SILR TG- | Charge Control | Amouphous Silica | 10 |
| 810G | Agent | (negative charge) | |
| | | | 505 |

TABLE 29
Formula 5-0% Recycled Content-Reformulation Baseline (+)
Composition fused to the paper but did not print-believed to be
due to the addition of the positive silica.

| Raw Material | Material Type | Description | Pounds |
|---|---|---|---|
| T-382-ES | Resin | Bis-A/Polyester | 385 |
| Black Pearls L A67 | Pigment | Carbon Black- virgin | 105 |
| Cab-o-SILR TG- | Charge Control | Amouphous Silica | 10 |
| 820F | Agent | (positive charge) | |
| | | | 500 |

TABLE 30
Formula 6 - 21% Recycled Content-Carbon Black
Composition printed and fused to the paper.

| Raw Material | Material Type | Description | Pounds |
|---|---|---|---|
| T-382-ES | Resin | Bis-A/Polyester | 385 |
| APEX CM powder | Pigment | Recycled Carbon black | 105 |
| Cab-o-SILR | Charge Control | Amouphous Silica | 10 |
| TG-820F | Agent | (positive charge) | |
| | | | 500 |

TABLE 31
Formula 7 - 0% Recycled Content-No Silica Charge Agents
Composition fused to the paper but did not print-believed to be due to the
lack of charge agents in the composition.

| Raw Material | Material Type | Description | Pounds |
|---|---|---|---|
| T-382-ES | Resin | Bis-A/Polyester | 390 |
| Black Pearls L A67 | Pigment | Carbon Black- virgin | 110 |
| | | | 500 |

TABLE 32
Formula 8 - 22% Recycled Content-Carbon Black,
No Charge Control Agent
Composition printed and fused to the paper.

| Raw Material | Material Type | Description | Pounds |
|---|---|---|---|
| T-382-ES | Resin | Bis-A/Polyester | 390 |
| APEX CM powder | Pigment | Recycled Carbon black | 110 |
| | | | 500 |

TABLE 33
Formula 9 - 30% Recycled Content-PET Oligomers
Composition fused to the paper but did not print-believed to be
due to the lack of charge agents in the composition.

| Raw Material | Material Type | Description | Pounds |
|---|---|---|---|
| T-6694 | Resin | Bis-A Epoxy | 240 |
| Digested Polyester | Resin | Digested Polyester (KF) | 150 |
| Black Pearls L A67 | Pigment | Carbon Black- virgin | 110 |
| | | | 500 |

TABLE 34
Formula 10 - 52% Recycled Content-No Charge Agents
Composition printed and fused to the paper.

| Raw Material | Material Type | Description | Pounds |
|---|---|---|---|
| T-6694 | Resin | Bis-A Epoxy | 240 |
| Digested | Resin | Digested Polyester (KF) | 150 |
| Polyester | | | |
| APEX CM powder | Pigment | Recycled Carbon black | 110 |
| | | | 500 |

TABLE 35
Formula 11 - 30% Hypothetical Recycled Content - Aldrich PET Trimer
Composition printed and fused to the paper.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| Black Pearls L A67 | Pigment | Carbon Black- virgin | 105 |
| Cab-o-SILR TG-810G | Charge Control | Amouphous Silica | 10 |
| | Agent | (negative charge) | |
| | | | 505 |

TABLE 36
Formula 12 - 51% Hypothetical Recycled Content -
Aldrich PET Trimer and Carbon Black (1)
Composition printed and fused to the paper.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| APEX CM powder | Pigment | Recycled Carbon black | 105 |
| Cab-o-SILR TG-810G | Charge Control | Amouphous Silica | 10 |
| | Agent | (negative charge) | |
| | | | 505 |

TABLE 37
Formula 13 - 30% Hypothetical Recycled Content -
Aldrich PET Trimer (+)
Composition would not attempt to print -
toner cartridge/printer issue.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| Black Pearls L A67 | Pigment | Carbon Black- virgin | 105 |
| Cab-o-SILR TG-820F | Charge Control | Amouphous Silica | 10 |
| | Agent | (positive charge) | |
| | | | 505 |

TABLE 38
Formula 15(*) - 52% Hypothetical Recycled Content -
Aldrich PET Trimer, No Charge Agents
Composition fused to the paper, but did not print-believed
to be due to the lack of charge agents in the composition.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| Black Pearls L A67 | Pigment | Carbon Black- virgin | 110 |
| | | | 500 |

(*) Formula 14 did not provide sufficient material for processing.

TABLE 39
Formula 16 - 52% Hypothetical Recycled Content -
Aldrich PET Trimer and Carbon Black
Composition printed and fused to the paper.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| APEX CM powder | Pigment | Recycled Carbon black | 110 |
| | | | 500 |

TABLE 40
Formula 17 - 30% Hypothetical Recycled Content -
Aldrich PET Trimer-Reheology Modified Trime
Composition printed and fused to the paper.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| Black Pearls L A67 | Pigment | Carbon Black- virgin | 105 |
| T-RM-12 | Rheology | Bis-A Epoxy | 5 |
| | Modifier | | |
| | | | 500 |

TABLE 41
Formula 18 - 53% Hypothetical Recycled Content - Aldrich PET
Trimer, Rheology Modified Trimer and Carbon Black
Composition printed and fused to the paper.

| Raw Material | Material Type | Description | Pounds |
|---|---|---|---|
| T-6694 | Resin | Bis-A Epoxy | 240 |
| Bis(2-hydroxyethyl) | Resin | Trimer PET: 465151 | 150 |
| terpthalate | | | |
| APEX CM powder | Pigment | Recycled Carbon black | 105 |
| T-RM-12 | Rheology | Bis-A Epoxy | 5 |
| | Modifier | | |
| | | | 500 |

TABLE 42
Jet Milling and Particle Size

| Formula | Rate | PSI | Temp | Particle Size/Remarks | Weights |
|---|---|---|---|---|---|
| 1 | Raw | | | Light gray granular powder (less carbon) - not screened | |
| 1 | 5 pph | 115 | Amb | 5.25μ D50 15.16μ D100 | 444 g |
| 2 | Raw | | | Black dusty powder (more carbon) - not screened | |
| 2 | 5 pph | 115 | Amb | 6.61μ D50 19.89μ D100 | 432 g |
| 5 | Raw | | | Medium gray larger granular mix - screened <10 mesh | |
| 5 | 5 pph | 115 | Amb | 5.88μ D50 19.88μ D100 | 447 g |
| 6 | Raw | | | Gray granules with large white clumps - screened <10 mesh | |
| 6 | 5 pph | 115 | Amb | 5.16μ D50 15.16μ D100 | 445 g |
| 7 | Raw | | | Dark gray + black granules up 1/32″ - screened <10 mesh | |
| 7 | 5 pph | 115 | Amb | 4.93μ D50 13.23μ D100 | 462 g |
| 8 | Raw | | | Black fine granular - not screened | |
| 8 | 5 pph | 115 | Amb | 5.34μ D50 15.16μ D100 | 443 g |
| 11 | Raw | | | Gray granular with very hard ½″ chunks - screened <10 mesh | |
| 11 | 5 pph | 115 | Amb | 4.37μ D50 13.23μ D100 | 440 g |
| 12 | Raw | | | Black powder with ¼″ chunks - screened <10 mesh | |
| 12 | 5 pph | 115 | Amb | 4.04μ D50 11.56μ D100 | 434 g |
| 13 | Raw | | | Gray powder with ¼″ chunks - screened <10 mesh | |
| 13 | 5 pph | 115 | Amb | 4.22μ D50 11.55μ D100 | 388 g |
| 15 | Raw | | | Dark gray powder with very hard ½″ chunks - screened <10 mesh | |
| 15 | 5 pph | 115 | Amb | 4.40μ D50 13.28μ D100 | 448 g |
| 16 | Raw | | | Black fine powder with hard ½″ chunks - screened <10 mesh | |
| 16 | 5 pph | 115 | Amb | 4.40μ D50 13.24μ D100 | 443 g |
| 17 | Raw | | | Black granular with many ½″ chunks - screened <10 mesh | |
| 17 | 5 pph | 115 | Amb | 4.12μ D50 11.55μ D100 | 459 g |
| 18 | Raw | | | Dark gray granules with black ½″ chunks - screened <10 mesh | |
| 18 | 5 pph | 115 | Amb | 4.70μ D50 15.15μ D100 | 467 g |
| 3 | Raw | | | Gray granules with 1″ white sticky clumps - screened <10 mesh | |
| 3 | 5 pph | 115 | Amb | 4.04μ D50 11.55μ D100 | 288 g |
| 4 | Raw | | | Light gray granules with 1″ white sticky clumps - blinded screen | |
| 4 | 5 pph | 115 | Amb | 12.02μ D50 88.49μ D100 Mill plugged up-test stopped. | 73 g |
| 9 | Raw | | | Black granules with 3″ white sticky balls - screened <10 mesh | |
| 9 | 5 pph | 115 | Amb | 7.92μ D50 26.08μ D100 | 395 g |
| 10 | Raw | | | Black granules with 3″ white sticky balls - screened <10 mesh | |
| 10 | 5 pph | 115 | Amb | 6.37μ D50 22.76μ D100 White material not as sticky | 410 g |

In order to test the uniformity of particle charging, experiments comparing the charge control were performed. The results of these experiments are summarized in Table 43. In particular, toner compositions comprising (a) carbon black (both recycled and virgin), (b) silica (both positive and negative), (c) magnetite (both natural and synthetic) and (d) digested rPET oligomers produced according to the processes described herein above, were analyzed. It was found that ideally the charge control agent (i.e., magnetite) is on the inside of the molecule, while the charging agent (i.e., silica) is on the outside of the molecule.

It was also determined that trace minerals residing within the recycled carbon black led to uneven charging and thereby degraded the quality of the toner.

A control sample (23A) and two test samples (23B and 23C) were sent to B&P Processing of 1000 Hess Ave, Saginaw, Mich. 48601 for extrusion. The materials were extruded and pelletized. The extrusion process confirmed that the digested rPET material was able to be extruded and successfully blended to form a uniform mixture. The samples were then sent to The Jet Pulverizer Co. of 1255 North Church Street Moorestown, N.J. 08057-1166 for milling. The samples were milled to the particle size of 8 microns, a size desirable for toner applications. It should be noted that all samples only required a single pass through the mill in order to reach 8 microns.

TABLE 43

| | Resin | | | | | | | |
|---|---|---|---|---|---|---|---|---|
| | and | | | | | | | |
| | DPET | Extrusion | | Synthetic | Synthetic | | | |
| | extruded | Conditions | | Fe (g) | Fe (%) | (+) silica | (+) silica (%) | Total (g) |
| 23A | 500 | 50-75 | RPM 160 F | 0 | 0 | 0 | 0 | 500 |
| 23B | 484 | 50-75 | RPM 160 F | 100.05 | 16.81371313 | 11 | 1.848584153 | 595.05 |
| 23C | 178.5 | 100 | RPM 200 F | 20.32 | 10.01034534 | 4.17 | 2.054288389 | 202.99 |

Magnetic jump toners use magnetite, a ferrimagnetic mineral with a chemical formula of Fe₃O₄, one of several iron oxides and a member of the spinel group, to deposit toner onto paper. A commercial toner produced by HP (i.e., HP Black Toner Cartridge for 4000 and 4050 Series) was subjected to thermogravimetric analysis (TGA) in order to determine the amount of iron/magnetite present in the composition. It was determined that the TGA product by HP contained 47.6% of magnetite.

In order to test the magnetic printing ability of a toner composition containing oligomeric digested rPET produced according to the above-described processes, several samples were prepared with different amount of digested rPET material, rheology modifier (t-6694, produced by Reichhold of World Headquarters & Technology center located at Research Triangle Park, 2400 Ellis Road, Durham N.C. 27703 USA), toner resin (T-RM 12, produced by Reichhold of World Headquarters & Technology center located at Research Triangle Park, 2400 Ellis Road, Durham N.C. 27703 USA), and natural or synthetic magnetite for study against a control (Formula 8). The addition of a rheology modifier with the extruded samples was used to lower the Glass Transition Temperature of the extruded resins.

The extrusion was processed at C.W Brabender Instruments, Inc. of 50 East Wesley Street P.O. Box 2127 South Hackensack, N.J. 07606. The extrusion was performed on a TSE20 Twin-Screw Compounder (Clamshell design, co-rotating screws, segmented elements) with a DDSR20 Twin-Screw BRABENDER Feeder (TC20/05 screws) and a Single Strand Die with 1/16″ Nozzle Insert, Conveyor and Pelletizer. All samples were run between 45-125 degrees Celsius. (In Table 45, the number or range of numbers following KFN ### is the extrusion temperate or range. Ex KF8 110 is formula 8 at 110 degrees Celsius). The extrusion rheometry was monitored, stored and printed using the WINEXT® software program by BRABENDER. The samples extruded were able to be strung and pelletized with no issue. A summary of the results of these samples is given in Table 44 and the milling information for the magnetic jump toner is given in Table 45. The percent recycled content was based on the total toner composition; however 47.2% of each toner formulation is inorganic iron.

TABLE 44

| | % | % | % | % | Extruded | Recycled |
|---|---|---|---|---|---|---|
| | Digested | t-6694 | T-RM 12 | Fe | (yes/no) | (%) |
| Formula | 20 | 32.8 | | 47.2 | Yes | 20 |
| KF1 | | | | | | |
| Formula | 40 | 12.8 | | 47.2 | No | 40 |
| KF2 | | | | | | |
| Formula | 20 | 27.8 | 5 | 47.2 | Yes | 20 |
| KF3 | | | | | | |
| Formula | 30 | 17.8 | 5 | 47.2 | Yes | 30 |
| KF4 | | | | | | |
| Formula | 40 | 7.8 | 5 | 47.2 | No | 40 |
| KF5 | | | | | | |
| Formula | 25 | 17.8 | 10 | 47.2 | Yes | 25 |
| KF6 | | | | | | |
| Formula | 30 | 6.9 | 15.9 | 47.2 | Yes | 30 |
| KF7 | | | | | | |
| Formula | | 52.8 | | 47.2 | Yes | 0 |
| KF8 | | | | | | |

TABLE 45

| | Mean Value | | | |
|---|---|---|---|---|
| Sample | μm | D50 μm | D90 μm | D100 μm |
| KF8 100 Second Pass | 6.1 | 5.9 | 10.7 | 18.9 |
| KF8 125 Second Pass | 7.7 | 7.5 | 12.8 | 20.7 |
| KF8 100B Second Pass | 5.2 | 5.4 | 8.6 | 11.8 |
| KF4 60B Third Pass | 13.8 | 10.4 | 30.6 | 76.4 |
| KF4 60 Second Pass | 36.0 | 30.7 | 76.8 | 121.8 |
| KF4 60 110 Third Pass | 14.5 | 13.6 | 26.2 | 43.7 |
| KF3 85-70 Third Pass | 18.6 | 15.6 | 37.4 | 83.9 |
| KF3 125-100 Third Pass | 32.1 | 27.5 | 67.6 | 101.1 |
| KF3 85-110 Third pass | 31.2 | 25.0 | 63.2 | 194.2 |
| KF6 45 Third Pass | 30.4 | 18.6 | 74.8 | 213.2 |

| | |
|---|---|
| KF6 60 | Did not grind. |

Both toners prepared for magnetic jump and electrostatic deposition laser printers printed and properly fused to the paper.

### Physical Testing of the Magnetic and Electrostatic Toner Printed Images

Samples were tested for crease degradation by ASTM F 1351. As a comparative test, ASTM F 1351 can be used to determine the damage caused by creasing paper, that is, damage to paper, coatings or images affixed to the paper and the loss of image quality and legibility that can result from creasing. Specimens were rated for pass or fail and if the coating was removed by creasing the image. Each sample was creased in the same area by rolling a 1 kg weight across a bent edge of a sample sheet of printed and folded paper.

Samples were also tested for removal of coating by a modified version of ASTM 3359 cross cut tape adhesion. As a comparative test, ASTM 3359 can be used to determine the removal of toner by applying tape to the printed surface. The tape is secured and then removed at a 90 degree angle from the paper. The tape is positioned on the image and the amount of image degradation is determined. Specimens were rated on the amount of toner removed. The results of the crease and cross cut tape adhesion tests are summarized in Table 46.

TABLE 46

| Formula and | | | Crease | Tape | |
|---|---|---|---|---|---|
| Number of | Fused | Printed | (Pass/ | Adhe- | |
| Page Tested | (Y/N) | (Y/N) | Fail) | sion | Comments |
| F1 page 50 | Y | Y | Pass | Very Low | No visual |
| | | | | | change in |
| | | | | | image |
| F2 page 100 | Y | Y | Pass | None | |
| F3 page 50 | Y | Y | Not | Not | Charging |
| (approx) | | | Tested | Tested | Seemed to |
| | | | (NT) | (NT) | be Reversed |
| F5 page 50 | Y | Y | NT | NT | Charging |
| (approx) | | | | | Seemed to |
| | | | | | be Reversed |
| F6 page 100 | Y | Y | Pass | Small | |
| (approx) | | | | amount | |
| | | | | removed | |
| F7 page 50 | Y | Y | NT | NT | |
| (approx) | | | | | |
| F8 page 100 | Y | Y | Pass | Very little | |
| | | | | removed | |
| F9 page 50 | Y | X | NT | NT | |
| (approx) | | | | | |
| F10 page 100 | Y | Y | Pass | Very little | Printing is |
| (approx) | | | | removed | light |
| F11 page 100 | Y | Y | Pass | Little | Printing is |
| (approx) | | | | removed | light |
| F12 page 100 | Y | Y | Pass | Very Little | |
| | | | | Removed | |
| F15 page 47 | Y | X | NT | NT | |
| F16 page 100 | Y | Y | Pass | Little | Removed |
| (approx) | | | | removed | significant |
| | | | | | amount at |
| | | | | | crease |
| F17 page 100 | Y | Y | Pass | Very Little | |
| (approx) | | | | Removal | |
| F18 page 75 | Y | Y | Pass | Small | |
| (approx) | | | | Removal | |
| 23A page 200 | Y | N | NT | NT | Charging |
| (approx) | | | | | Seemed to |
| | | | | | be Reversed |
| 23B page 100 | N | N | NT | NT | Extremely |
| (approx) | | | | | light printing |
| | | | | | (possibly |
| | | | | | from residual |
| | | | | | commercial |
| | | | | | toner |
| KF8-125 page 25 | Y | Y | Pass | Very little | Printing is |
| (approx) | | | | removed | light |
| KF8-100 B | Y | Y | Pass | Very light | |
| Page 50(approx) | | | | Removed | |
| KF4 60-110 | Y | N-Y | NT | NT | Prints with |
| Page 25 (approx) | | | | | black specs |
| KF4 60 Page 50 | Y | Y | Pass | Little | |
| (approx) | | | | Removed | |
| KF8 100 Page 50 | Y | Y | Pass | Very little | Printing |
| (approx) | | | | to none | Very light |
| | | | | removed | |
| KF3 85-110 | Y | Y | Pass | None | Good quality |
| Page 150 | | | | | print |
| Control (MI 486) | Y | Y | Pass | None | Light printing |

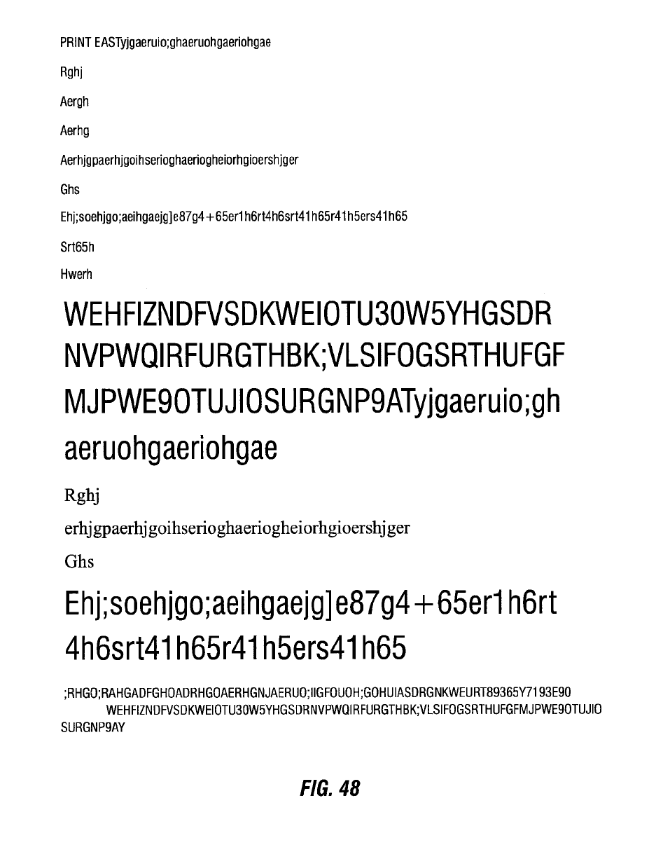

All sample toners (either prepared for the magnetic jump printer or the electrostatic deposition laser printer) printed and properly fused to the paper. The result of a printing experiment is shown in FIG. 48 wherein the toner composition comprised KF3 85 which was printed on an HP LaserJet 4050.

Digested rPET oligomers produced from rPET may, as has been demonstrated, be used in toners as polyester resin replacements and/or extenders. These digested rPET oligomers from rPET may be of different molecular weight distributions. That is, the molecular weight distributions of the digested rPET oligomers from rPET may be very sharp (i.e., have a polydispersity value approaching 1.0) so as to have essentially only one type structure present such as a trimer in which terephthalic acid is endcapped with ethylene glycol. Or the molecular weight distributions of the digested rPET oligomers from rPET may be more broad (i.e., have a polydispersity value receding from 1.0) which represents several types of structures blended together such as one or more trimers, tetramers, pentamers, etc. It is believed that a digested rPET oligomer having an N value greater than or equal to 2 to about 1000 is acceptable for use with the presently disclosed and claimed inventive concept(s). Different properties are tailorable for each type of structure or N value of digested rPET oligomeric material used.

Digested rPET oligomers from rPET are also useful as chain extenders for polyester and epoxy type resins. Mixed polyesters and polyester modified epoxy resins are thus possible. Such materials can be used in a variety of applications including coatings and graphic arts media. Also these digested rPET oligomers from rPET are reactive with isocyanates to produce many new polyurethanes for use in coatings and foams. Furthermore, digested rPET oligomers from rPET can be reacted with epichlorohydrin to produce new epoxy type materials. These new epoxy materials are reactive and can be used in normal epoxy type applications.

What I claim is:

1. An oligomeric form of polyethylene terephthalate having an average molecular weight (MW) of from about 250 to about 26,535.

2. A method for making an oligomeric form of polyethylene terephthalate, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
reacting the polyethylene terephthalate, glycolysis reagent and the catalyst to thereby provide an oligomeric form of polyethylene terephthalate having a molecular weight less than the initial molecular weight.

3. The method of claim 2, wherein the glycolysis reagent is ethylene glycol.

4. The method of claim 3, wherein the ethylene glycol is present in a molar ratio of from 1:1 to about 5:1 with respect to the polyethylene terephthalate.

5. The method of claim 2, wherein the polyethylene terephthalate is at least 20 percent recycled content.

6. The method of claim 5, wherein the polyethylene terephthalate is obtained from 100 percent recycled content.

7. The method of claim 2, wherein the polyethylene terephthalate is clear.

8. The method of claim 2, wherein the polyethylene terephthalate is green.

9. The method of claim 8, further comprising the step of removing any residual green color in the oligomeric form of polyethylene terephthalate produced.

10. The method of claim 2, wherein the polyethylene terephthalate comprises a mixture of green and clear polyethylene terephthalate.

11. The method of claim 10, further comprising the step of removing any residual green color in the oligomeric form of polyethylene terephthalate produced.

12. The method of claim 2, wherein:
the polyethylene terephthalate has an initial average molecular weight (MW) greater than or equal to 10,000; and
the glycolysis reagent comprises an oligomeric form of polyethylene terephthalate having an average molecular weight (MW) less than or equal to 10,000.

13. The method of claim 2, wherein the catalyst is a zinc acetate catalyst.

14. The method of claim 2, wherein the method is performed at atmospheric pressure.

15. The method of claim 2, wherein the polyethylene terephthalate has an average molecular size greater than or equal to 25 microns.

16. The method of claim 2, wherein the polyethylene terephthalate has an average molecular size greater than or equal to 100 microns.

17. The method of claim 2, wherein the polyethylene terephthalate has an average molecular size greater than or equal to 200 microns.

18. A method for making a mixture of oligomeric forms of polyethylene terephthalate, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide a mixture of oligomeric forms of polyethylene terephthalate having a molecular weight less than the initial molecular weight, wherein the polydispersity of the mixture of oligomeric forms of polyethylene terephthalate is less than or equal to 1.5.

19. A mixture of oligomeric forms of polyethylene terephthalate wherein the polydispersity of the mixture is less than or equal to 1.5.

20. A mixture of oligomeric forms of polyethylene terephthalate wherein the polydispersity of the mixture is less than or equal to 2.0.

21. A mixture of oligomeric forms of polyethylene terephthalate wherein the polydispersity of the mixture is less than or equal to 2.5.

22. A method of making an oligomeric form of polyethylene terephthalate having an average molecular weight (MW) greater than or equal to 10,000, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
providing means for directing the molecular weight of an oligomeric form of polyethylene terephthalate produced from the reaction of the polyethylene terephthalate, glycolysis reagent and the catalyst, wherein the average molecular weight (MW) of the produced oligomeric form of polyethylene terephthalate is greater than or equal to 10,000.

23. A method of making an oligomeric form of polyethylene terephthalate having an average molecular weight (MW) less than or equal to 15,000, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
providing means for directing the molecular weight of an oligomeric form of polyethylene terephthalate produced from the reaction of the polyethylene terephthalate, glycolysis reagent and the catalyst, wherein the molecular weight (MW) of the produced oligomeric form of polyethylene terephthalate is less than or equal to 15,0000.

24. A method of making an oligomeric form of polyethylene terephthalate having an average molecular weight (MW) less than or equal to 5,000, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
providing means for directing the molecular weight of an oligomeric form of polyethylene terephthalate produced from the reaction of the polyethylene terephthalate, glycolysis reagent and the catalyst, wherein the average molecular weight (MW) of the produced oligomeric form of polyethylene terephthalate is less than or equal to 5,000.

25. A method for decreasing the molecular weight (MW) of polyethylene terephthalate, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide an oligomeric form of polyethylene terephthalate having a molecular weight less than the initial molecular weight.

26. A method for making an oligomeric form of polyethylene terephthalate, comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide an oligomeric form of polyethylene terephthalate having a molecular weight less than the initial molecular weight, wherein at least a portion of the glycolysis reagent is an amount of an oligomeric form of polyethylene terephthalate.

27. A glycolysis reagent comprising an effective amount of an oligomeric form of polyethylene terephthalate sufficient to decrease the molecular weight of polyethylene terephthalate in the presence of a reaction catalyst.

28. An article comprising an effective amount of an oligomeric form of polyethylene terephthalate having an average molecular weight (MW) of from about 250 to about 10,000, wherein the article is produced according to the method comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide an oligomeric form of polyethylene terephthalate having an average molecular weight (MW) of from about 250 to about 10,000.

29. An article comprising an effective amount of an oligomeric form of polyethylene terephthalate having an average molecular weight (MW) of from about 250 to about 10,000, wherein the article is produced according the method comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide an oligomeric form of polyethylene terephthalate having an average molecular weight (MW) of from about 250 to about 10,000, wherein the article comprises at least 20 percent recycled content.

30. A toner composition for use in a laser printer, comprising an effective amount of an oligomeric form of polyethylene terephthalate having an average molecular weight (MW) of from about 250 to about 10,000, wherein the article is produced according to the method comprising the steps of:
introducing an amount of polyethylene terephthalate having an initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide an oligomeric form of polyethylene terephthalate having an average molecular weight (MW) of from about 250 to about 10,000, wherein the article comprises at least 20 percent recycled content.

31. A method for making an oligomeric form of polyethylene terephthalate, comprising the steps of:
introducing an amount of polyethylene terephthalate having an average initial molecular weight into a reaction vessel;
introducing an amount of a glycolysis reagent into the reaction vessel;
introducing an amount of a catalyst into the reaction vessel; and
allowing the polyethylene terephthalate, glycolysis reagent and the catalyst to react and thereby provide an oligomeric form of polyethylene terephthalate having an average molecular weight less than the initial molecular weight, wherein the glycolysis reagent comprises at least in part a recycled portion of an oligomeric form of polyethylene terephthalate previously produced.
